(19) United States
(12) Patent Application Publication
(10) Pub. No.: US 2007/0197603 A1
(43) Pub. Date: Aug. 23, 2007

(54) Substituted indole ligands for the orl-1 receptor

(76) Inventors: Alessandra Consonni, Milan (IT); Simone Del Sordo, Milan (IT); Carlo Farina, Milan (IT); Stefania Gagliardi, Milan (IT); Silvano Ronzoni, Milan (IT); Stefania Vallese, Milan (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/561,282

(22) PCT Filed: Jul. 1, 2004
(86) PCT No.: PCT/EP04/07294

§ 371 Date: Feb. 1, 2007

(30) Foreign Application Priority Data
Jul. 4, 2003 (IT) MI2003A001378
Jul. 4, 2003 (IT) MI2003A001379

(51) Int. Cl. A61K 31/44; C07D 401/06

(52) U.S. Cl. 514/339; 546/201

(57) ABSTRACT

New ligands for the ORL-1 receptor are described, useful for modulating the activity of said receptors in a patient in need thereof, and for preventing and treating illnesses dependent on the stimulation of this receptor. The new compounds conform to structural formula (I), 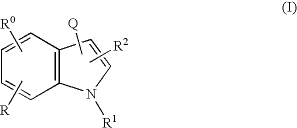
wherein Q is a moiety of formula: 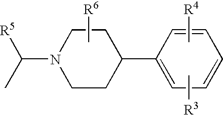
and R, R⁰, R¹, R², R³, R⁴, R⁵, R⁶ are further defined in the description.

The present invention relates to certain new compounds, a process for their preparation, to pharmaceutical compositions which contain them, and to the use of these compounds in medicine. The invention relates in particular to a group of new compounds possessing antagonistic or agonistic activity for the receptors ORL-1 and useful in treating illness related to modulation of these receptors.

The ORL-1 receptor is located along the entire neural axis and is involved in various pathological phenomena, including the transmission of pain. Various peptide and non-peptide ligands for the ORL-1 receptor are known; the non-peptide ligands include known compounds with morphinan, benzimidazopiperidine, spiropiperidine, arylpiperidine and 4-aminoquinoline structure (Life Sciences, 73, 2003, 663-678). WO 0183454 and WO 03040099 describe other ORL-1 antagonists with benzosuberonylpiperidine structure substituted in position 5 by a hydroxy, alkoxy, amino or alkylamino group, and their synthesis method. J. Med. Chem., 1997, 40(23), 3912-14 and WO 9709308 describe certain indoles substituted in position 3 with a dipiperazine group, as antagonists for the receptor NPY-1. J. Med. Chem., 1996, 39(10), 1941-2, WO 9424105, WO 9410145, WO 02241894, WO9629330 and GB 2076810 describe variously substituted 3-piperazinylmethyl indoles as ligands for dopamine receptors, in particular for the D4 receptor. GB 2083476 describes specific 3-arylpiperidinylmethyl indoles as 5HT uptake inhibitors. U.S. Pat. No. 5,215,989 describes certain di-substituted piperazine and imidazole derivatives useful as class III antiarrhythmic agents. EP 846683 describes hydroxypiperidine derivatives as NMDA receptor blockers. WO 200241894 describes 2-piperazino substituted indoles, useful as antagonists for the dopamine D4 receptors. GB 1063019 describes certain piperidinoalkyl substituted indoles, useful as myorelaxants.

It has now been found that certain substituted indoles, i.e indoles substituted in position 2 or 3 with a 4-arylpiperidinoalkyl group, are powerful ligands for the ORL-1 receptor, and can therefore be useful in the treatment and/or prevention of diseases dependent on the modulation of this receptor. They can thus be used in man or animals for treating and/or preventing pain, gastrointestinal disorders, diseases of the immune system, dysfunctions of the cardiovascular system, diseases of the excretory system, sexual dysfunction, disorders of the respiratory tract, central nervous system disorders, drug abuse, tolerance and dependence, etc. Examples of specific diseases dependent on the modulation of the ORL-1 receptor are listed further on in this specification.

The compounds of the invention conform to structural formula (I), 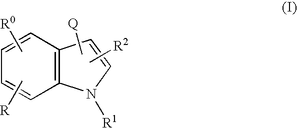
or a pharmaceutically acceptable salt or solvate thereof, wherein:
R and R⁰ are each independently hydrogen, halogen, C₁₋₆alkyl, perhaloC₁₋₆alkyl, C₁₋₆alkoxy, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl, cyano and, when R and R⁰ are on adjacent carbon atoms, methylenedioxy and ethylenedioxy;
R¹ is hydrogen, C₁₋₆alkyl, C₃₋₆alkenyl, C₃₋₆alkinyl, arylC₁₋₆alkyl, heteroarylC₁₋₆alkyl, (C₃₋₇cycloalkyl)alkyl, aminoC₁₋₆alkyl, (C₁₋₆alkyl)aminoC₁₋₆alkyl, di(C₁₋₆alkyl)aminoC₁₋₆alkyl, hydroxyC₁₋₆alkyl, C₁₋₆alkoxyC₁₋₆alkyl, aryloxyC₁₋₆alkyl, CO-aryl, SO₂aryl, aryl, C₁₋₆alkoxycarbonylC₁₋₆alkyl, where each aryl or heteroaryl can be substituted one or more times by halogen, C₁₋₆alkoxy, C₁₋₆alkyl, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl or perhaloC₁₋₆alkyl;
R² is C₃₋₇cycloalkyl, aryl, heteroaryl, arylC₁₋₆alkyl, heteroarylC₁₋₆alkyl, C₁₋₆alkoxycarbonyl, hydroxyC₁₋₆alkyl, aminocarbonyl, C₁₋₆alkylaminocarbonyl, di(C₁₋₆alkyl)aminocarbonyl where each aryl or heteroaryl can be substituted one or more times by halogen, C₁₋₆alkoxy, C₁₋₆alkyl, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl or perhaloC₁₋₆alkyl;
Q is a moiety of formula: 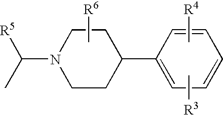
wherein:
R³ and R⁴ are each independently hydrogen, halogen, C₁₋₆alkyl, perhaloC₁₋₆alkyl, C₁₋₆alkoxy, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl;
R⁵ is hydrogen or C₁₋₆alkyl, and
R⁶ is hydrogen or hydroxymethyl.

In said formula (I),

R and R⁰ are preferably, hydrogen, halogen, C₁₋₆alkyl, C₁₋₆alkoxy; more preferably, R and R⁰ are hydrogen, chlorine, fluorine, methyl, methoxy.

R¹ is preferably hydrogen, C₁₋₆alkyl, C₃₋₆alkenyl, C₃₋₆alkinyl, arylC₁₋₆alkyl, (C₃₋₇cycloalkyl)alkyl, hydroxyC₁₋₆alkyl, CO-aryl, SO₂-aryl; more preferably, R¹ is hydrogen, methyl, n-propyl, isopentyl, allyl, 2-hydroxyethyl, cyclopropylmethyl, cyclohexylmethyl, benzyl, fluorobenzyl, chlorobenzyl, bromobenzyl, methoxybenzyl, methylbenzyl, t-butylbenzyl, trifluoromethylbenzyl, diphenylmethyl, phenoxyethyl, 2-naphthylmethyl, benzoyl, benzenesulfonyl.

R² is preferably aryl, heteroaryl, arylC₁₋₆alkyl, C₁₋₆alkoxycarbonyl; more preferably, R² is phenyl, chlorophenyl, methoxyphenyl, fluorophenyl, 2-furyl, 2-thienyl, 2-pyridyl, benzyl, ethoxycarbonyl.

R³ and R⁴ are preferably hydrogen, halogen, C₁₋₆alkyl, perhaloC₁₋₆alkyl, C₁₋₆alkoxy; more preferably, R³ and R⁴ are hydrogen, chlorine, fluorine, bromine, methyl, methoxy, trifluoromethyl.

R⁵ is preferably hydrogen. R⁶ is preferably hydrogen.

The term “aryl” as used herein includes the C₅₋₁₀aryl groups, in particular phenyl and naphthyl. The C₁₋₆alkyl groups can be linear or branched and are preferably C₁₋₂alkyl groups, more preferably methyl. The term “halogen” includes the iodine, chlorine, bromine and fluorine groups, especially chlorine, fluorine and bromine.

The term “heteroaryl” includes saturated and unsaturated heterocyclic rings. Preferred compounds of formula (I) according to the present invention include the following compounds or pharmaceutically acceptable salts or solvates thereof,
3-[4-(2,6-Dichloro-phenyl)-piperidin-1-ylmethyl]-2-phenyl-1H-indole
3-[4-(2,6-Dichloro-phenyl)-piperidin-1-ylmethyl]-1H-indole
3-[4-(2,6-Dichloro-phenyl)-piperidin-1-ylmethyl]-2-methyl-1H-indole
2-(4-Chloro-phenyl)-3-[4-(2,6-dimethyl-phenyl)-piperidin-1-ylmethyl]-1H-indole
2-Phenyl-3-[4-(2-trifluoromethyl-phenyl)-piperidin-1-ylmethyl]-1H-indole
3-[4-(2,6-Dimethyl-phenyl)-piperidin-1-ylmethyl]-2-phenyl-1H-indole
2-Phenyl-3-(4-phenyl-piperidin-1-ylmethyl)-1H-indole
2-(2-Chloro-phenyl)-3-[4-(2,6-dichloro-phenyl)-piperidin-1-ylmethyl]-1H-indole
2-(2-Chloro-phenyl)-3-[4-(2,6-dimethyl-phenyl)-piperidin-1-ylmethyl]-1H-indole
2-(2-Chloro-phenyl)-3-[4-(2-trifluoromethyl-phenyl)-piperidin-1-ylmethyl]-1H-indole
2-(2-Chloro-phenyl)-3-(4-phenyl-piperidin-1-ylmethyl)-1H-indole
3-[4-(2,6-Dichloro-phenyl)-piperidin-1-ylmethyl]-2-(2-methoxy-phenyl)-1H-indole
3-[4-(2,6-Dimethyl-phenyl)-piperidin-1-ylmethyl]-2-(2-methoxy-phenyl)-1H-indole
2-(2-Methoxy-phenyl)-3-[4-(2-trifluoromethyl-phenyl)-piperidin-1-ylmethyl]-1H-indole
2-(2-Methoxy-phenyl)-3-(4-phenyl-piperidin-1-ylmethyl)-1H-indole
3-[4-(2,6-Dichloro-phenyl)-piperidin-1-ylmethyl]-2-(3-methoxy-phenyl)-1H-indole
3-[4-(2,6-Dimethyl-phenyl)-piperidin-1-ylmethyl]-2-(3-methoxy-phenyls-1H-indole
2-(3-Methoxy-phenyl)-3-[4-(2-trifluoromethyl-phenyl)-piperidin-1-ylmethyl]-1H-indole
2-(3-Methoxy-phenyl)-3-(4-phenyl-piperidin-1-ylmethyl)-1H-indole
2-(4-Chloro-phenyl)-3-[4-(2,6-dichloro-phenyl)-piperidin-1-ylmethyl]-1H-indole
2-(4-Chloro-phenyl)-3-[4-(2-trifluoromethyl-phenyl)-piperidin-1-ylmethyl]-1H-indole
2-(4-Chloro-phenyl)-3-(4-phenyl-piperidin-1-ylmethyl)-1H-indole
3-[4-(2,6-Dichloro-phenyl)-piperidin-1-ylmethyl]-2-(4-fluoro-phenyl)-1H-indole
3-[4-(2,6-Dimethyl-phenyl)-piperidin-1-ylmethyl]-2-(4-fluoro-phenyl)-1H-indole
2-(4-Fluoro-phenyl)-3-[4-(2-trifluoromethyl-phenyl)-piperidin-1-ylmethyl]-1H-indole
2-(4-Fluoro-phenyl)-3-(4-phenyl-piperidin-1-ylmethyl)-1H-indole
3-[4-(2,6-Dichloro-phenyl)-piperidin-1-ylmethyl]-2-furan-2-yl-1H-indole
3-[4-(2,6-Dimethyl-phenyl)-piperidin-1-ylmethyl]-2-furan-2-yl-1H-indole
2-Furan-2-yl-3-[4-(2-trifluoromethyl-phenyl)-piperidin-1-ylmethyl]-1H-indole
2-Furan-2-yl-3-(4-phenyl-piperidin-1-ylmethyl)-1H-indole
3-[4-(2,6-Dichloro-phenyl)-piperidin-1-ylmethyl]-2-pyridin-2-yl-1H-indole
3-[4-(2,6-Dimethyl-phenyl)-piperidin-1-ylmethyl]-2-pyridin-2-yl-1H-indole
2-Pyridin-2-yl-3-[4-(2-trifluoromethyl-phenyl)-piperidin-1-ylmethyl]-1H-indole
3-(4-Phenyl-piperidin-1-ylmethyl)-2-pyridin-2-yl-1H-indole
3-[4-(2,6-Dichloro-phenyl)-piperidin-1-ylmethyl]-2-thiophen-2-yl-1H-indole
3-[4-(2,6-Dimethyl-phenyl)-piperidin-1-ylmethyl]-2-thiophen-2-yl-1H-indole
2-Thiophen-2-yl-3-[4-(2-trifluoromethyl-phenyl)-piperidin-1-ylmethyl]-1H-indole
3-(4-Phenyl-piperidin-1-ylmethyl)-2-thiophen-2-yl-1H-indole
2-Benzyl-3-[4-(2,6-dimethyl-phenyl)-piperidin-1-ylmethyl]-1H-indole
2-Benzyl-3-[4-(2,6-dichloro-phenyl)-piperidin-1-ylmethyl]-1H-indole
3-[4-(4-Methoxy-phenyl)-piperidin-1-ylmethyl]-2-phenyl-1H-indole
3-[4-(2-Fluoro-phenyl)-piperidin-1-ylmethyl]-2-phenyl-1H-indole
3-[4-(3-Fluoro-phenyl)-piperidin-1-ylmethyl]-2-phenyl-1H-indole
3-[4-(4-Fluoro-phenyl)-piperidin-1-ylmethyl]-2-phenyl-1H-indole
2-Phenyl-3-[4-(4-trifluoromethyl-phenyl)-piperidin-1-ylmethyl]-1H-indole
3-[4-(2-Chloro-phenyl)-piperidin-1-ylmethyl]-2-phenyl-1H-indole
3-[4-(3-Chloro-phenyl)-piperidin-1-ylmethyl]-2-phenyl-1H-indole
3-[4-(4-Chloro-phenyl)-piperidin-1-ylmethyl]-2-phenyl-1H-indole
2-Phenyl-3-(4-o-tolyl-piperidin-1-ylmethyl)-1H-indole
3-[4-(2-Bromo-phenyl)-piperidin-1-ylmethyl]-2-phenyl-1H-indole
3-[4-(2,3-Dichloro-phenyl)-piperidin-1-ylmethyl]-2-phenyl-1H-indole
3-[4-(2,5-Dimethyl-phenyl)-piperidin-1-ylmethyl]-2-phenyl-1H-indole
3-[4-(2,6-Difluoro-phenyl)-piperidin-1-ylmethyl]-2-phenyl-1H-indole
3-[4-(3-Bromo-phenyl)-piperidin-1-ylmethyl]-2-phenyl-1H-indole
3-[4-(2-Methoxy-phenyl)-piperidin-1-ylmethyl]-2-phenyl-1H-indole
3-[4-(2,6-Dichloro-phenyl)-piperidin-1-ylmethyl]-5-fluoro-2-phenyl-1H-indole
3-[4-(2,6-Dimethyl-phenyl)-piperidin-1-ylmethyl]-1H-indole
3-[4-(2,6-Dimethyl-phenyl)-piperidin-1-ylmethyl]-2-methyl-1H-indole
Cis-[4-Phenyl-1-(2-phenyl-1H-indol-3-ylmethyl)-piperidin-3-yl]-methanol
Trans-[4-Phenyl-1-(2-phenyl-1H-indol-3-ylmethyl)-piperidin-3-yl]-methanol
5-Chloro-3-[4-(2,6-dimethyl-phenyl)-piperidin-1-ylmethyl]-2-phenyl-1H-indole
3-[4-(2,6-Dimethyl-phenyl)-piperidin-1-ylmethyl]-5-methoxy-2-phenyl-1H-indole
7-[4-(2,6-Dimethyl-phenyl)-piperidin-1-ylmethyl]-6-phenyl-5H-[1,3]dioxolo[4,5-f]indole
3-[4-(2,6-Dimethyl-phenyl)-piperidin-1-ylmethyl]-1-(2-hydroxy-ethyl)-2-phenyl-1H-indol-5-ol
7-Bromo-3-[4-(2,6-dimethyl-phenyl)-piperidin-1-ylmethyl]-2-methyl-1H-indole;
3-[4-(2,6-Dimethyl-phenyl)-piperidin-1-ylmethyl]-5-fluoro-2-methyl-1H-indole;
3-[4-(2,6-Dimethyl-phenyl)-piperidin-1-ylmethyl]-5-fluoro-2-phenyl-1H-indole
3-[4-(2,6-Dichloro-phenyl)-piperidin-1-ylmethyl]-1H-indole-2-carboxylic acid ethyl ester
3-[4-(2,6-Dimethyl-phenyl)-piperidin-1-ylmethyl]-2-phenyl-1H-indole-6-carbonitrile
3-[4-(2,6-Dimethyl-phenyl)-piperidin-1-ylmethyl]-1,2-diphenyl-1H-indole
3-[4-(2,6-Dimethyl-phenyl)-piperidin-1-ylmethyl]-5-fluoro-1H-indole-2-carboxylic acid amide trifluoroacetate
3-{1-[4-(2,6-Dimethyl-phenyl)-piperidin-1-yl]-ethyl)-1H-indole
{3-[4-(2,6-Dichloro-phenyl)-piperidin-1-ylmethyl]-1H-indol-2-yl}-methanol
1-Benzyl-3-[4-(2,6-dichloro-phenyl)-piperidin-1-ylmethyl]-2-phenyl-1H-indole
3-[4-(2,6-Dichloro-phenyl)-piperidin-1-ylmethyl]-2-phenyl-1-propyl-1H-indole
3-[4-(2,6-Dichloro-phenyl)-piperidin-1-ylmethyl]-1-methyl-2-phenyl-1H-indole
1-Benzyl-3-[4-(2,6-dichloro-phenyl)-piperidin-1-ylmethyl]-5-fluoro-2-phenyl-1H-indole
1-Benzyl-3-[4-(2,6-dichloro-phenyl)-piperidin-1-ylmethyl]-2-methyl-1H-indole
1-Benzyl-3-[4-(2,6-dichloro-phenyl)-piperidin-1-ylmethyl]-1H-indole
1-Benzyl-3-[4-(2,6-dimethyl-phenyl)-piperidin-1-ylmethyl]-2-methyl-1H-indole
1-Benzyl-3-[4-(2,6-dimethyl-phenyl)-piperidin-1-ylmethyl]-2-phenyl-1H-indole
1-Benzyl-3-[4-(2,6-dimethyl-phenyl)-piperidin-1-ylmethyl]-1H-indole
1-Benzyl-5-chloro-3-[4-(2,6-dimethyl-phenyl)-piperidin-1-ylmethyl]-2-phenyl-1H-indole
1-Benzyl-3-[4-(2,6-dimethyl-phenyl)-piperidin-1-ylmethyl]-5-methoxy-2-phenyl-1H-indole
5-Benzyl-7-[4-(2,6-dimethyl-phenyl)-piperidin-1-ylmethyl]-6-phenyl-5H-[1,3]dioxolo[4,5-f]indole
{3-[4-(2,6-Dichloro-phenyl)-piperidin-1-ylmethyl]-5-fluoro-2-phenyl-indol-1-yl}-acetic acid methyl ester
3-(4-(2,6-Dichloro-phenyl)piperidin-1-ylmethyl)-1-(2-hydroxyethyl)-2-phenyl-1H-indole
2-{3-[4-(2,6-Dichloro-phenyl)-piperidin-1-ylmethyl]-5-fluoro-2-phenyl-indol-1-yl}-ethanol
2-{3-[4-(2,6-Dichloro-phenyl)-piperidin-1-ylmethyl]-indol-1-yl}-ethanol
2-{3-[4-(2,6-Dimethyl-phenyl)-piperidin-1-ylmethyl]-indol-1-yl}-ethanol
2-{3-[4-(2,6-Dimethyl-phenyl)-piperidin-1-ylmethyl]-2-methyl-indol-1-yl}-ethanol
2-{3-[4-(2,6-Dichloro-phenyl)-piperidin-1-ylmethyl]-2-methyl-indol-1-yl}-ethanol
2-{3-[4-(2,6-Dimethyl-phenyl)-piperidin-1-ylmethyl]-2-phenyl-indol-1-yl}-ethanol
3-{3-[4-(2,6-Dimethyl-phenyl)-piperidin-1-ylmethyl]-2-phenyl-indol-1-yl}-propan-1-ol
2-(3-[4-(2,6-Dimethyl-phenyl)-piperidin-1-ylmethyl]-5-methoxy-2-phenyl-indol-1-yl}-ethanol
2-{5-Chloro-3-[4-(2,6-dimethyl-phenyl)-piperidin-1-ylmethyl]-2-phenyl-indol-1-yl}-ethanol
2-{7-[4-(2,6-Dimethyl-phenyl)-piperidin-1-ylmethyl]-6-phenyl-[1,3]dioxolo[4,5-f]indol-5-yl}-ethanol
2-{3-[4-(2,6-Dimethyl-phenyl)-piperidin-1-ylmethyl]-5-fluoro-2-methyl-indol-1-yl}-ethanol
1-(4-tert-Butyl-benzyl)-3-[4-(2,6-dichloro-phenyl)-piperidin-1-ylmethyl]-2-phenyl-1H-indole trifluoroacetate
3-[4-(2,6-Dichloro-phenyl)-piperidin-1-ylmethyl]-1-(3-methyl-butyl)-2-phenyl-1H-indole
1-Cyclopropylmethyl-3-[4-(2,6-dichloro-phenyl)-piperidin-1-ylmethyl]-2-phenyl-1H-indole
3-[4-(2,6-Dichloro-phenyl)-piperidin-1-ylmethyl]-1-(3-methoxy-benzyl)-2-phenyl-1H-indole
3-[4-(2,6-Dichloro-phenyl)-piperidin-1-ylmethyl]-1-(2-methyl-benzyl)-2-phenyl-1H-indole
1-Cyclohexylmethyl-3-[4-(2,6-dichloro-phenyl)-piperidin-1-ylmethyl]-2-phenyl-1H-indole
3-[4-(2,6-Dichloro-phenyl)-piperidin-1-ylmethyl]-1-(4-methyl-benzyl)-2-phenyl-1H-indole
3-[4-(2,6-Dichloro-phenyl)-piperidin-1-ylmethyl]-1-(4-fluoro-benzyl)-2-phenyl-1H-indole
1-(3-Chloro-benzyl)-3-[4-(2,6-dichloro-phenyl)-piperidin-1-ylmethyl]-2-phenyl-1H-indole trifluoroacetate
1-(2-Chloro-benzyl)-3-[4-(2,6-dichloro-phenyl)-piperidin-1-ylmethyl]-2-phenyl-1H-indole
1-(4-Chloro-benzyl)-3-[4-(2,6-dichloro-phenyl)-piperidin-1-ylmethyl]-2-phenyl-1H-indole
1-Allyl-3-[4-(2,6-dichloro-phenyl)-piperidin-1-ylmethyl]-2-phenyl-1H-indole
3-[4-(2,6-Dichloro-phenyl)-piperidin-1-ylmethyl]-2-phenyl-1-prop-2-ynyl-1H-indole
3-[4-(2,6-Dichloro-phenyl)-piperidin-1-ylmethyl]-1-(2-methoxy-benzyl)-2-phenyl-1H-indole trifluoroacetate
3-[4-(2,6-Dichloro-phenyl)-piperidin-1-ylmethyl]-1-(4-methoxy-benzyl)-2-phenyl-1H-indole trifluoroacetate
1-(4-Bromo-benzyl)-3-[4-(2,6-dichloro-phenyl)-piperidin-1-ylmethyl]-2-phenyl-1H-indole trifluoroacetate
1-Biphenyl-4-ylmethyl-3-[4-(2,6-dichloro-phenyl)-piperidin-1-ylmethyl]-2-phenyl-1H-indole trifluoroacetate
3-[4-(2,6-Dichloro-phenyl)-piperidin-1-ylmethyl]-1-naphthalen-2-ylmethyl-2-phenyl-1H-indole trifluoroacetate
3-[4-(2,6-Dichloro-phenyl)-piperidin-1-ylmethyl]-1-(2-phenoxy-ethyl)-2-phenyl-1H-indole trifluoroacetate
3-[4-(2,6-Dichloro-phenyl)-piperidin-1-ylmethyl]-1-(3-methyl-benzyl)-2-phenyl-1H-indole trifluoroacetate
3-[4-(2,6-Dichloro-phenyl)-piperidin-1-ylmethyl]-1-(2-fluoro-benzyl)-2-phenyl-1H-indole trifluoroacetate
3-[4-(2,6-Dichloro-phenyl)-piperidin-1-ylmethyl]-1-(3-fluoro-benzyl)-2-phenyl-1H-indole trifluoroacetate
3-[4-(2,6-Dichloro-phenyl)-piperidin-1-ylmethyl]-2-phenyl-1-(2-trifluoromethyl-benzyl)-1H-indole trifluoroacetate
3-[4-(2,6-Dichloro-phenyl)-piperidin-1-ylmethyl]-2-phenyl-1-(3-trifluoromethyl-benzyl)-1H-indole trifluoroacetate
3-[4-(2,6-Dichloro-phenyl)-piperidin-1-ylmethyl]-2-phenyl-1-(4-trifluoromethyl-benzyl)-1H-indole trifluoroacetate
1-Benzenesulfonyl-3-[4-(2,6-dichloro-phenyl)-piperidin-1-ylmethyl]-2-phenyl-1H-indole trifluoroacetate
1-Benzoyl-3-[4-(2,6-dichloro-phenyl)-piperidin-1-ylmethyl]-2-phenyl-1H-indole trifluoroacetate
2-[4-(2,6-Dichloro-phenyl)-piperidin-1-ylmethyl]-1H-indole
2-[4-(2,6-Dichloro-phenyl)-piperidin-1-ylmethyl]-3-methyl-1H-indole
2-[4-(2,6-Dimethyl-phenyl)-piperidin-1-ylmethyl]-1H-indole
2-[4-(2,6-Dichloro-phenyl)-piperidin-1-ylmethyl]-3-phenyl-1H-indole
2-[4-(2-Chloro-6-fluoro-phenyl)-piperidin-1-ylmethyl]-1H-indole
2-[4-(2,6-Dimethyl-phenyl)-piperidin-1-ylmethyl]-3-methyl-1H-indole
3-Methyl-2-(4-phenyl-piperidin-1-ylmethyl)-1H-indole
3-Phenyl-2-(4-phenyl-piperidin-1-ylmethyl)-1H-indole
3-Phenyl-2-(4-(3-trifluoromethylphenyl)piperidin-1-ylmethyl)-1H-indole
2-[4-(2,6-Dimethyl-phenyl)-piperidin-1-ylmethyl]-3-phenyl-1H-indole
2-(4-Phenyl-piperidin-1-ylmethyl)-1H-indole
2-[4-(2-Trifluoromethyl-phenyl)-piperidin-1-ylmethyl]-1H-indole
2-[4-(3-Trifluoromethyl-phenyl)-piperidin-1-ylmethyl]-1H-indole
2-[4-(4-Trifluoromethyl-phenyl)-piperidin-1-ylmethyl]-1H-indole
2-[4-(3-Fluoro-2-methyl-phenyl)-piperidin-1-ylmethyl]-1H-indole
5,6-Dichloro-2-[4-(2,6-dichloro-phenyl)-piperidin-1-ylmethyl]-1H-indole
5,6-Dichloro-2-[4-(2,6-dimethyl-phenyl)-piperidin-1-ylmethyl]-1H-indole
1-Benzyl-2-[4-(2,6-dichloro-phenyl)-piperidin-1-ylmethyl]-1H-indole
2-[4-(2,6-Dichloro-phenyl)-piperidin-1-ylmethyl]-1-propyl-1H-indole
2-[4-(2,6-Dichloro-phenyl)-piperidin-1-ylmethyl]-1-methyl-1H-indole
2-(4-(2,6-Dichlorophenyl)-piperidin-1-ylmethyl)-1-(2-hydroxyethyl)-1H-indole; and
1-Benzoyl-2-[4-(2,6-dichloro-phenyl)-piperidin-1-ylmethyl]-1H-indole.

The compounds of formula (I) can exhibit stereoisomerism because of the presence of chiral atoms and/or multiple bonds. The present invention therefore extends to stereoisomers of the compounds of the formula (I), including racemates, enantiomers, diastereoisomers and geometric isomers.

It has been found that, when a compound of formula (I) exhibits optical isomerism, an enantiomer possesses a greater affinity for the ORL-1 receptor than its antipod.

Consequently, the present invention also provides an enantiomer of a compound of formula (I).

In a further aspect, the present invention provides a mixture of enantiomers of a compound of formula (I) where an enantiomer is present in a proportion greater than its antipod.

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labelled reagent.

The present invention also provides processes for preparing the compounds of formula (I).

In its general embodiment, the process is characterised by the step of reacting a compound of formula (III) 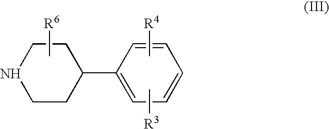
wherein R³, R⁴, R⁶ are as defined as in the aforedescribed formula (I), with a compound of formula (VII), 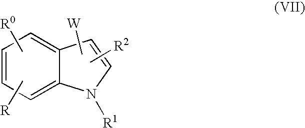
wherein R, R⁰, R¹, R², are as defined as in the aforedescribed formula (I), and W is hydrogen or a group capable of binding to the piperidinic nitrogen of said compound of formula (III), e.g. a carbonyl-containing group such as formyl, acyl, or carboxyl group.

When W is hydrogen and is in position 3, the reaction between (VII) and (III) is typically a Mannich reaction, taking place in an organic solvent environment, in presence of a suitable aldehydic reagent carrying the moiety R⁵, and acetic acid.

When W is formyl, acyl or carboxyl, the compound resulting from the reaction of (VII) with (III) is further treated with a reducing agent, thus obtaining said compound of formula (I); alternatively, when W is an acyl group, the reaction of (VII) with (III) can be performed under reductive amination conditions, obtaining in this case directly said compound of formula (I).

Said processes can be performed both on N-substituted or N-unsubstituted indoles of formula (VII); in the latter case, final compounds of formula (I) where R¹ is other than hydrogen can be suitably obtained by known means, i.e. by reacting said unsubstituted indoles of formula (VII) or any derivatives thereof, with a reagent of formula R¹—X, where R¹ has the meanings defined above, and X is a suitable leaving group. As known in the art, the reaction with R¹—X may take place e.g. in basic conditions, or under phase transfer conditions.

More specifically, the compounds of formula (I) in which R¹ is hydrogen, R² is in position 2 and Q is in position 3 on the indole ring, hereinafter referred as formula (Ia), can be obtained as follows: a compound of formula (IIa) 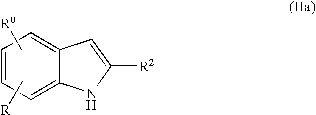
in which R, R⁰ and R² have the meanings given for formula (I), is functionalised in position 3 by reaction with an aldehydic derivative of formula CH(R⁵)O where R⁵ is as above defined and a compound of formula (III) 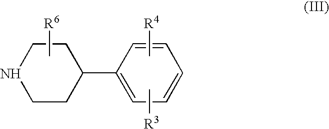
where R³, R⁴ and R⁶ are as defined for formula (I), obtaining compounds of formula (Ia).

The functionalization reaction is preferably a Mannich reaction, as described in standard reference texts of synthesis methodologies such as J. March, Advanced Organic Chemistry, 3^{rd} Edition (1985), Wiley Interscience. In particular, the compounds of formula (Ia) can be prepared in accordance with scheme 1, starting from compounds of formula (IIa), said derivative of formula CH(R⁵)O, and amines of formula (III).

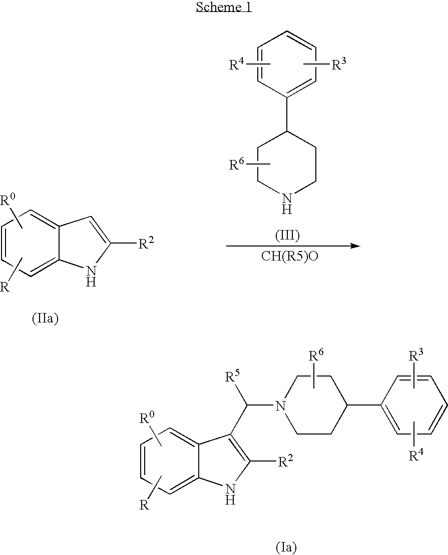

In a typical procedure, an amine of formula (III) is dissolved in a suitable solvent, such as for example methanol or dioxane or a mixture of both, to which solution the derivative of formula CH(R⁵)O and acetic acid are added. After a suitable time, typically between 5 min and 1 h, there is added to the preceding solution a solution of an indole of formula (IIa) in a suitable solvent, such as for example methanol or dioxane or a mixture of both, while maintaining the temperature of the resultant solution generally between 0° C. and ambient temperature. The reaction mixture is stirred for a suitable time, typically between 1 h and 96 h, at a suitable temperature, typically between 0° C. and 80° C., after which it is processed by known methods.

Two preferred processing procedures are here indicated as procedure A and procedure B.

In procedure A, water is added to the reaction mixture followed by a solution of a suitable base, such as aqueous ammonium hydroxide, until basic pH is reached, after which it is extracted with a suitable organic solvent such as ethyl acetate. The organic phase is collected and dried with, for example, sodium sulfate, and the solvent is removed by evaporation. The crude product can be purified, if necessary, by conventional purification methods such as flash chromatography, trituration, crystallization or preparative HPLC.

In procedure B, the reaction mixture is poured onto an acid resin cartridge and eluted with a suitable solvent, such as for example dichloromethane or methanol, to remove non-basic impurities, and then with a solution of a suitable base in a suitable organic solvent such as, for example, a methanolic ammonia solution, to recover the desired compound of formula (I). The solvent is removed by evaporation and the crude product can be purified, if necessary, by conventional purification methods such as flash chromatography, trituration, crystallization or preparative HPLC.

The compounds of formula (I) in which R¹ is hydrogen, R² is in position 3 and Q is in position 2 on the indole ring, hereinafter referred as formula (Ib), can be obtained by treating an aldehyde or a ketone of formula (IIb) 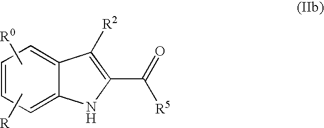
in which R, R⁰, R² and R⁵ have the meanings given for formula (I), with a compound of formula (III) 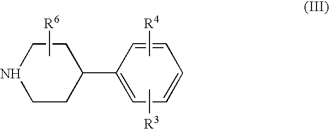
where R³, R⁴ and R⁶ are as defined for formula (I).

The treatment of compounds of formula (IIb) with (III) normally takes place under reductive amination conditions, for example in an environment of sodium cyanoborohydride in methanol (Lane, Synthesis, 135, 1975). This reaction, illustrated hereinafter in scheme 2, leads directly to the obtaining of the final compound of formula (Ib).

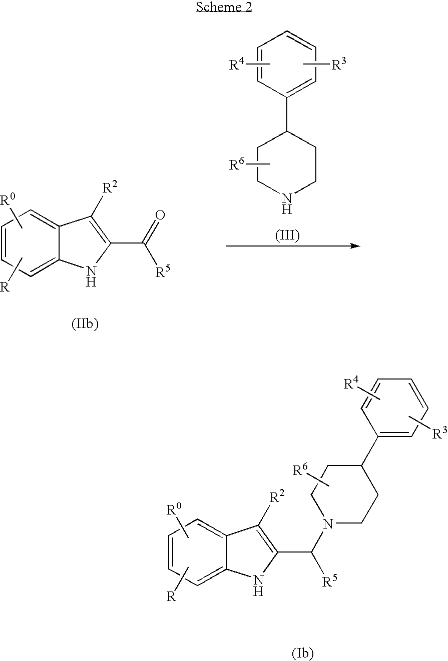

In a typical procedure, a solution of an amine of formula (III) in a suitable solvent such as methanol or ethanol is added to a solution of compound of formula (IIb) in a suitable solvent such as methanol or ethanol. After stirring for a suitable time, typically between 30 min and 6 h, a suitable reducing agent is added such as sodium cyanoborohydride, followed by acetic acid; if necessary, further additions of reducing agent can be made. After stirring for a suitable time, typically between 8 h and 24 h, the solvent is evaporated and the resultant residue is taken up with a suitable solvent, such as ethyl acetate; water is added, and after exhaustive extraction the organic phase is recovered, dried with for example sodium sulfate and the solvent is removed by evaporation. The crude product can be purified, if necessary, by conventional purification methods such as flash chromatography, trituration and preparative HPLC.

According to an alternative typical procedure, a reducing agent supported on polymer such as cyanoborohydride supported on polymer, a solution of a compound of formula (IIb) and an amine of formula (III) as free base or corresponding salt in a suitable organic solvent such as methanol, tetrahydrofuran or a mixture of both, and acetic acid, are mixed at a suitable temperature, typically ambient temperature, for a suitable time, typically between 15 h and 60 h; if amine salts of formula (III) are used, a solution of sodium acetate in a suitable solvent such as methanol can be added to the initial reaction mixture. The polymer is then filtered off and washed with a suitable solvent such as methanol or tetrahydrofuran. The resultant solution can be concentrated, if necessary, and then poured onto a cartridge of acid resin and eluted with a suitable solvent such as methanol, to remove non-basic impurities, and then with a solution of a suitable base in a suitable organic solvent such as a methanolic solution of ammonia, to recover the desired compound of formula (Ib). The solvent is removed by evaporation and the crude product can be purified, if necessary, by conventional purification methods such as flash chromatography, trituration and preparative HPLC.

The compounds of formula (IIa) and (IIb) are known or commercially available compounds or can be prepared by procedures described in standard reference texts of synthesis methodologies such as J. March, Advanced Organic Chemistry, 3^{rd} Edition (1985), Wiley Interscience.

The compounds of formula (III) are known or commercially available or can be prepared by the procedures described in WO 01/83454.

The compounds of formula (Ib) wherein R⁵ is hydrogen can also be obtained by treating an acid of formula (IVb) 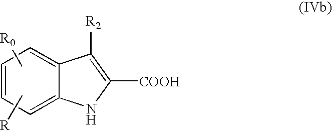
in which R, R⁰ and R² have the meanings given for formula (I), with a compound of formula (III) 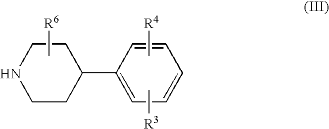
where R³, R⁴ and R⁶ are as defined for formula (I).

The reaction of acids of formula (IVb) with (III) leads to the formation of the corresponding amide (Vb), as indicated hereinafter in scheme 3; the final compound of formula (Ib) is obtained by subsequent reduction of the carbonyl group of the amide by conventional methods, e.g. with a suitable reducing agent such as a metal hydride or a reducing agent containing borane.

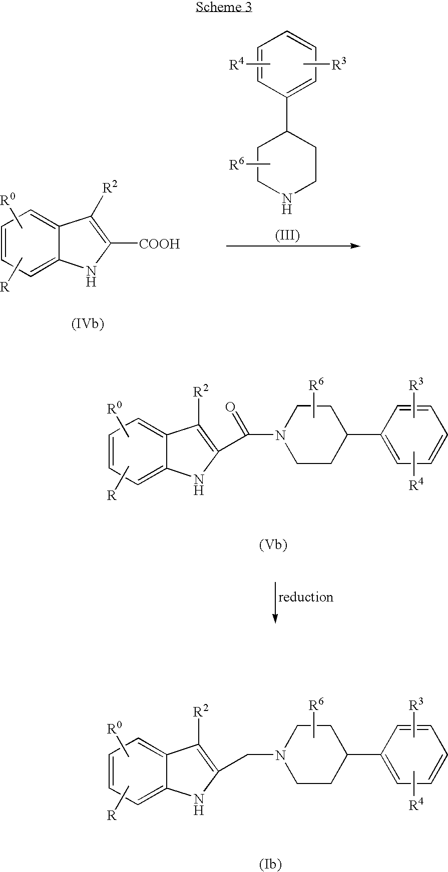

The compounds of formula (Vb) can be prepared by reacting the suitably activated compound of formula (IVb) with the amine of formula (III).

Activation of the compound of formula (IVb), effected before reacting with the compounds of formula (III), can suitably take place by forming the corresponding acyl halides, for example by reaction with oxalyl chloride or thionyl chloride; alternatively, the compounds of formula (IVb) can also be activated in situ, i.e. in the presence of a compound of formula (III), using activating agents such as dicyclohexylcarbodiimide/1-hydroxybenzotriazole, N-ethyl-N′-(3-dimethylamino-propyl)carbodiimide/1-hydroxybenzotriazole, or O-benzotriazol-1-yl-N,N,N′,N′-tetramethyluronium hexafluorophosphate.

In a typical procedure, a compound of formula (IVb) is dissolved in a suitable solvent such as tetrahydrofuran or dichloromethane, and activating agents, such as N-ethyl-N′-(3-dimethylaminopropyl)-carbodiimide and 1-hydroxybenzotriazole are added to this solution at a suitable temperature, typically between 0° C. and ambient temperature. After a suitable time, typically between 30 min and 12 h, to this solution there is added a solution of an amine of general formula (III) in a suitable solvent such as tetrahydrofuran or dichloromethane, at a suitable temperature typically between 0° C. and ambient temperature. After a suitable time, typically between 1 h and 48 h, water is added and the reaction mixture is extracted with a suitable solvent such as ethyl acetate or dichloromethane. The organic phase is recovered, dried, with for example sodium sulfate, and the solvent is removed by evaporation. The crude product can be purified, if necessary, by conventional purification methods such as flash chromatography, trituration and preparative HPLC.

In the second step illustrated in scheme 3, the amide (Vb) is reduced by reaction with known reducing agents such as a metal hydride or a reducing agent containing borane, to obtain the final compound (Ib).

In a typical procedure, the amide (Vb) is dissolved in a suitable solvent such as tetrahydrofuran in a suitable inert atmosphere, typically of argon or nitrogen, and a suitable reducing agent such as a borane-tetrahydrofuran complex is added; the addition is made at a suitable temperature, typically between 0° C. and ambient temperature. Said reaction mixture is stirred at a suitable temperature, typically between 0° C. and the reflux temperature, for a suitable time, typically between 1 h and 8 h. A suitable quenching agent, such as water or an aqueous acid or basic solution, is added to the reaction mixture at a suitable temperature, typically 0° C., and after agitation at a suitable temperature for a suitable time, the reaction mixture is extracted with a suitable solvent such as diethyl ether or ethyl acetate. The organic phase is recovered and dried with, for example, sodium sulfate, and the solvent is removed by evaporation. The crude product can be purified, if necessary, by conventional purification methods such as flash chromatography, trituration and preparative HPLC. The compounds of formula (IVb) are known or commercially available or can be prepared by procedures described in standard reference texts of synthesis methodologies such as J. March, Advanced Organic Chemistry, 3rd Edition (1985), Wiley Interscience.

The compounds of formula (I) in which R¹ is different from hydrogen, R² is in position 2 and Q is in position 3 on the indole ring, hereinafter referred as formula (Ic), can be obtained as follows: a compound of formula (IIa) 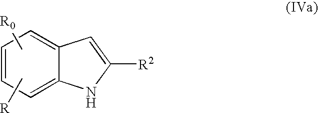
wherein R¹, R^{o} and R² have the meanings given for formula (I), is treated in accordance with the two following steps, which can take place in any order:
a) reaction with an aldehydic derivative of formula CH(R⁵)O where R⁵ is as above defined and a compound of formula (III) 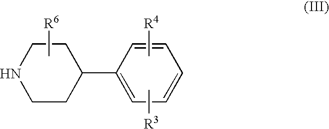
where R³ R⁴ and R⁶ are as defined for formula (I),
b) reaction with a compound of formula R¹—X, in which R¹ is as defined in formula (I) and X is a suitable leaving group,
thus obtaining the compounds of formula (Ic).

If the steps are carried out in the order (a)→(b), the compound of formula (IIa) is firstly functionalized in position 3 by reaction with said aldehydic derivative of formula CH(R⁵)O and the compound of formula (III); the 3-functionalized intermediate obtained is then N-alkylated in position 1 of the indole ring by treatment with the compound R¹—X, to obtain the final compound of formula (Ic).

If the steps are carried out in the reverse order (b)→(a), the compound of formula (IIa) is firstly N-alkylated in position 1 of the indole ring by reaction with the compound R¹—X; the N-alkylated intermediate obtained is then 3-functionalized by reaction with the said compound CH(R⁵)O and the compound of formula (III), to obtain the final compound of formula (Ic).

Step (a) (3-functionalization) is effected preferably by the Mannich reaction, in the previously detailed manner.

Step (b) is a nucleophilic reaction which can be effected by commonly known methods; in particular it is effected by reacting the compound of formula (IIa) (or, as illustrated in the following Scheme 4, its 3-substituted derivative resulting from step (a)) with a strong base and then treating the resultant indolyl anion with said compound of formula R¹—X.

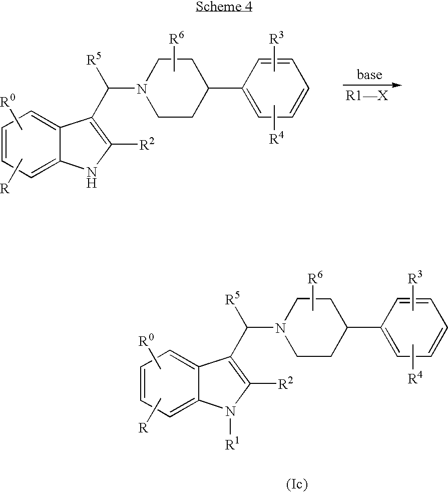

In a typical procedure, a suitable base such as sodium hydride, is added under an inert atmosphere, typically of argon or nitrogen, to a solution of a compound of formula (IIa) or its 3-substituted derivative in a suitable anhydrous solvent, such as dimethylformamide, at a suitable temperature, typically between 0° C. and ambient temperature. After a suitable time, typically between 15 min and 1 h, a suitable alkyl or acyl halide of formula R¹—X is added to the reaction mixture, either as such or dissolved in a suitable anhydrous solvent such as dimethylformamide; if necessary, further additions of alkyl or acyl halide can be made. The resultant reaction mixture is stirred at a suitable temperature, typically ambient temperature, for a suitable time, typically between 1 h and 20 h. The procedure can be carried out by known methods. Two preferred working procedures are here indicated as procedure A and procedure B.

In procedure A, water is added to the reaction mixture, which is then extracted with a suitable organic solvent such as diethylether. The organic phase is collected and dried with, for example, sodium sulfate, and the solvent is removed by evaporation. The crude product can be purified, if necessary, by conventional purification methods such as flash chromatography, trituration, crystallization and preparative HPLC.

In working procedure B, water is added to the reaction mixture, which is then filtered through a suitable water retention filter, eluting with a suitable solvent such as ethyl acetate. The resultant solution can be concentrated, if necessary, and then poured onto an acid resin cartridge and eluted with a suitable solvent, such as methanol, to remove non-basic impurities, and then with a solution of a suitable base in a suitable organic solvent such as a methanolic solution of ammonia, to recover the desired compound of formula (I). The solvent is removed by evaporation and the crude product can be purified, if necessary, by conventional purification methods such as flash chromatography, trituration, crystallization and preparative HPLC.

Alternatively, step (b) can be effected by reacting the compound of formula (IIa) (or, as illustrated in scheme 5, its 3-substituted derivative resulting from step (a)) with a suitable alkylating/acylating agent of formula R¹—X, under phase transfer conditions, as described in WE. Keller, Phase-Transfer Reactions, Vols. 1 e 2, 1986, Georg Thieme Verlag.

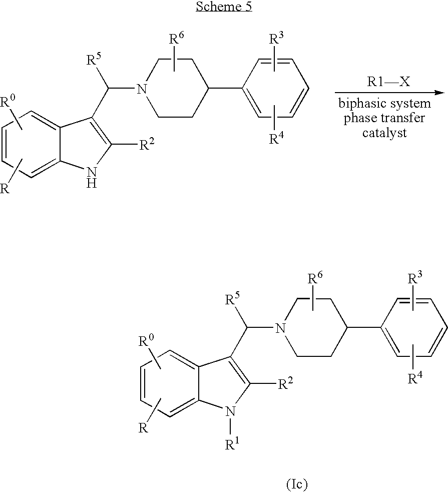

In a typical procedure, the compound of formula (IIa) or its 3-substituted derivative is dissolved in a suitable biphasic system, typically a 1:1 mixture of toluene and an aqueous solution of sodium hydroxide; a suitable phase-transfer catalyst, such as Aliquat® 336 is then added. After a suitable time, typically between 10 min and 1 h, a suitable alkyl- or acyl halide of formula R¹—X is added to the reaction mixture; if necessary, further additions of alkyl- or acyl halide can be made. The reaction mixture is stirred vigorously at a suitable temperature, typically ambient temperature, for a suitable time, typically from 5 h to 20 h, then filtered through a suitable water retention filter, eluting with a suitable solvent such as ethyl acetate. The solvent is removed by evaporation and the crude product can be purified, if necessary, by conventional purification methods such as flash chromatography, trituration, crystallization and preparative HPLC.

The compounds of formula R¹—X, for example alkylating/acylating agents, used in step (b), are known or commercially available, or can be prepared by procedures described in standard reference texts of synthesis methodologies such as J. March, Advanced Organic Chemistry, 3rd Edition (1985), Wiley Interscience.

The compounds of formula (I), in which R¹ is other than hydrogen, R² is in position 3 and Q is in position 2 on the indole ring, hereinafter referred as formula (Id), can be obtained by reacting the following derivatives (obtainable in accordance with the already illustrated schemes 2 and 3) in which R, R⁰, R², R³, R⁴, R⁵ and R⁶ are as previously defined for formula (I), with a compound of formula R¹—X, in which R¹ is as defined for formula (I) and is other than hydrogen, and X is a suitable leaving group: in this manner the desired compound of formula (Id) is obtained.

In a first embodiment, this reaction can take place as described in the following scheme 6, by reacting the compound of formula (Ib), with a strong base and then treating the resultant indolyl anion with a suitable alkylating/acylating agent of formula R¹—X.

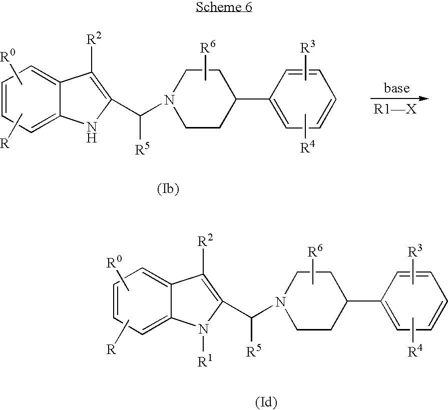

In a typical procedure, a suitable base, such as sodium hydride, is added under an inert atmosphere, typically of argon or nitrogen, to a solution of the starting compound in a suitable anhydrous solvent, such as dimethylformamide, at a suitable temperature, typically between 0° C. and ambient temperature. After a suitable time, typically between 15 min and 1 h, a suitable alkyl or acyl halide of formula R¹—X is added to the reaction mixture, either as such or dissolved in a suitable anhydrous solvent such as dimethylformamide; if necessary, further additions of alkyl or acyl halide can be made. The resultant reaction mixture is stirred at a suitable temperature, typically ambient temperature, for a suitable time, typically between 1 h and 20 h. The procedure can be carried out by known methods. Two preferred working procedures are here indicated as procedure A and procedure B.

In procedure A, water is added to the reaction mixture, which is then extracted with a suitable organic solvent such as diethylether. The organic phase is collected and dried with, for example, sodium sulfate, and the solvent is removed by evaporation. The crude product can be purified, if necessary, by conventional purification methods such as flash chromatography, trituration, crystallization and preparative HPLC.

In working procedure B, water is added to the reaction mixture, which is then filtered through a suitable water retention filter, eluting with a suitable solvent such as ethyl acetate. The resultant solution can be concentrated, if necessary, and then poured onto an acid resin cartridge and eluted with a suitable solvent, such as methanol, to remove non-basic impurities, and then with a solution of a suitable base in a suitable organic solvent such as a methanolic solution of ammonia, to recover the desired compound of formula (Id). The solvent is removed by evaporation and the crude product can be purified, if necessary, by conventional purification methods such as flash chromatography, trituration, crystallization and preparative HPLC.

In a second embodiment, the compounds of formula (Id) can be obtained as shown in scheme 7, by reacting a compound of formula (Ib) with a suitable alkylating/acylating agent of formula R¹—X under phase transfer conditions, as described in W. E. Keller, Phase-Transfer Reactions, Vols. 1 e 2, 1986, Georg Thieme Verlag.

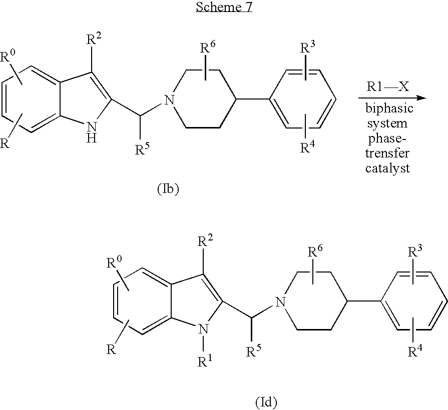

In a typical procedure, the starting compound is dissolved in a suitable biphasic system, such as a mixture of tetrahydrofuran or toluene or dichloromethane and an aqueous solution of sodium hydroxide, and a suitable phase-transfer catalyst, such as tetrabutylammonium bromide or hydrogen sulfate is added. After a suitable time, typically between 10 min and 1 h, a suitable alkyl- or acyl halide of formula R¹—X is added to the reaction mixture; if necessary, further additions of alkyl- or acyl halide can be made. The reaction mixture is stirred vigorously at a suitable temperature, typically between ambient temperature and 100° C., for a suitable time, typically from 5 h to 20 h, then water is added and the reaction mixture is extracted with a suitable solvent such as diethyl ether, ethyl acetate or dichloromethane. The organic phase is recovered and dried, for example, with sodium sulfate, and the solvent is removed by evaporation. The crude product can be purified, if necessary, by conventional purification methods such as flash chromatography, trituration, crystallization and preparative HPLC.

As aforestated, the compounds of formula (I) are ligands for the ORL-1 receptor. Hence, there is provided a compound of formula (I) for use as an active therapeutic substance.

According to another aspect of the present invention a method is provided for modulating the activity of the ORL-1 receptor in a human or animal patient in need thereof, comprising administering to the human or animal patient an effective quantity of a compound of formula (VI): formula (VI) is in all respects identical to formula (I) as herein described, with the sole difference that the meanings for R² also include hydrogen and C₁₋₆alkyl.

Another aspect of the present invention provides the use of a compound of formula (VI) in the manufacture of a medicament for human or animal administration, useful for modulating the activity of the ORL-1 receptor. Said compounds of formula (VI) can be agonists or antagonists of the ORL-1 receptor.

The compounds of the invention are therefore useful in the therapy and/or prophylaxis of all those illnesses dependent on modulation of the ORL-1 receptor. Accordingly they can be used as analgesics in man or animals in treating or preventing, for example, acute pain, chronic neuropathic or inflammatory pain, including post-herpes neuralgia, neuralgia, diabetic neuropathy and post-infarct pain; visceral pain including that associated with irritable bowel syndrome, dysmenorrhea, and hyperreflexia of the bladder; osteoarthritis, back pain, labour pain in childbirth.

Said compounds can further be useful in the treatment or prophylaxis of gastrointestinal disorders including irritable bowel syndrome, and symptoms associated with non-ulcerous dyspepsia and gastro-oesophageal reflux; diseases of the immune system; dysfunctions of the cardiovascular system such as infarct, congestive cardiac insufficiency and pathologies associated with alterations of arterial pressure; diseases of the excretory system, such as altered diuresis, water homeostasis and sodium excretion, syndrome of inappropriate anti-diuretic hormone secretion (SIADH); sexual dysfunctions including impotence and frigidity; cirrhosis with ascites.

These compounds can also be useful in the treatment or prophylaxis of disorders of the respiratory tract such as cough, asthma, adult respiratory distress syndrome (ARDS), altered pulmonary function, including chronic obstructive pulmonary disease.

Compounds of the invention are further useful in the treatment of central nervous system disorders where ORL-1 receptors are involved. In particular in the treatment or prevention of major depressive disorders including bipolar depression, unipolar depression, single or recurrent major depressive episodes with or without psychotic features, catatonic features, melancholic features, atypical features or postpartum onset, the treatment of anxiety and the treatment of panic disorders. The term anxiety includes anxiety disorders, such as panic disorders with or without agoraphobia, agoraphobia, phobias, for example, social phobias or agoraphobia, obsessive-compulsive disorder, stress disorders including post-traumatic stress disorders, generalised anxiety disorders, acute stress disorders and mixed anxiety-depression disorders. Other mood disorders encompassed within the term major depressive disorders include dysthymic disorder with early or late onset and with or without atypical features, neurotic depression, post traumatic stress disorders, post operative stress and social phobia; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood. Major depressive disorders may also result from a general medical condition including, but not limited to, myocardial infarction, diabetes, miscarriage or abortion, etc.

Compounds of the invention are also useful in the treatment or prevention of dementia as such, e.g. vascular dementia and dementia associated with AIDS; they are further effective in treating or preventing motor damage and neurodegeneration due to Alzheimer's disease; senile dementia, Parkinson's disease or other neurodegenerative pathologies.

Compounds of the invention are also useful in the treatment or prevention of epilepsy; schizophrenic disorders including paranoid schizophrenia, disorganised schizophrenia, catatonic schizophrenia, undifferentiated schizophrenia, residual schizoprenia.

Compounds of the invention are also useful for the treatment of dysfunction of appetite and food intake and in circumstances such as anorexia, anorexia nervosa bulimia and metabolic disorders such as obesity.

Compounds of the invention are also useful in the treatment of sleep disorders including dysomnia, insomnia, sleep apnea, narcolepsy, and circadian rhythmic disorders.

Compounds of the invention are also useful in the treatment or prevention of cognitive disorders. Cognitive disorders include dementia, amnestic disorders, memory loss, and cognitive disorders not otherwise specified. Furthermore compounds of the invention are also useful as memory and/or cognition enhancers in healthy humans with no cognitive and/or memory deficit. Compounds of the invention are also useful in the treatment of drug abuse, tolerance to and dependence on a number of substances. For example, they are useful in the treatment of dependence on nicotine, alcohol, caffeine, phencyclidine (phencyclidine like compounds), or in the treatment of tolerance to and dependence on opiates (e.g. cannabis, heroin, morphine) or benzodiazepines; in the treatment of cocaine, sedative ipnotic, amphetamine or amphetamine-related drugs (e.g. dextroamphetamine, methylamphetamine) addiction or a combination thereof.

As indicated above, the compounds of formula (I) can form salts, especially pharmaceutically acceptable salts. Suitable pharmaceutically acceptable salts are those used conventionally in the art and include those described in J. Pharm. Sci., 1977, 66, 1-19, such as acid addition salts.

Suitable pharmaceutically acceptable salts include acid addition salts.

Suitable pharmaceutically acceptable acid addition salts include salts with inorganic acids such, for example, as hydrochloric acid, hydrobromic acid, orthophosphoric acid or sulphuric acid, or with organic acids such, for example as methanesulphonic acid, toluenesulphonic acid, acetic acid, propionic acid, lacetic acid, citric acid, fumaric acid, malic acid, succinic acid, salicylic acid, maleic acid, glycerophosphoric acid or acetylsalicylic acid.

The salts and/or solvates of the compounds of the formula (I) which are not pharmaceutically acceptable may be useful as intermediates in the preparation of pharmaceutically acceptable salts and/or solvates of compounds of formula (I) or the compounds of the formula (I) themselves, and as such form another aspect of the present invention.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates. Solvates include stoichiometric solvates and non-stoichiometric solvates.

An effective quantity of compound of the invention depends on factors such as the nature or seriousness of the illness or illnesses to be treated and on the weight of the patient. In all cases a unit dose normally contains from 0.1 to 50 mg, for example from 0.5 to 10 mg, of the compound. Unit doses are normally administered one or more times per day, for example, 2, 3 or 4 times a day, in particular from 1 to 3 times per day, so that the total daily dose is normally, for an adult of 70 kg, between 0.1 and 50 mg, for example between 0.1 and 5 mg, i.e. in the approximate range of 0.001 to 1 mg/kg/day, in particular between 0.005 and 0.2 mg/kg/day. For oral or parenteral administration, it is highly preferred that the compound be administered in the form of unit dose composition for example, in the form of unit dose oral or parenteral composition.

These compositions are prepared by mixing and are suitably adapted to oral or parenteral administration, and as such can be in the form of tablets, capsules, oral preparations, powders, granules, lozenges, reconstitutable powders, injectable or infusible liquid solutions, suspensions or suppositories.

Tablets and capsules for oral administration are normally presented in unit dose form, and contain conventional excipients such as binders, fillers, diluents, compressing agents, lubricants, detergents, disintegrants, colorants, aromas and wetting agents. The tablets can be covered by methods well known in the art.

Suitable fillers include cellulose, mannitol, lactose and other similar agents. Suitable disintegrants include starch, polyvinylpyrrolidone and starch derivatives such as sodium glycolate starch. Suitable lubricants include, for example, magnesium stearate. Suitable wetting agents include sodium laurylsulfate.

These solid oral compositions can be prepared by conventional methods of mixing, filling or compression. The mixing operations can be repeated to disperse the active component in compositions containing large quantities of fillers. These operations are conventional.

Oral liquid preparations can be in the form, for example, of aqueous or oily suspensions, solutions, emulsions, syrups, or elixirs, or can be presented as a dry product for reconstitution with water or with a suitable carrier before use. These liquid preparations can contain conventional additives such as suspending agents, for example sorbitol, syrup, methylcellulose, gelatin, hydroxyethylcellulose, carboxymethylcellulose, aluminium stearate gel or hydrogenated edible fats, emulsifying agents, for example lecithin, sorbitan monooleate, or acacia; non-aqueous carriers (which can include edible oils), for example almond oil, fractionated coconut oil, oily esters such as glycerine esters, propylene glycol, or ethyl alcohol; preservatives, for example methyl or propyl p-hydroxybenzoate or sorbic acid, and if desired, conventional aromas or colorants.

Oral formulations also include conventional slow-release formulations, such as tablets or granules having an enteric coating.

For parenteral administration, fluid dose units can be prepared, containing the compound and a sterile carrier. The compound, depending on the carrier and the concentration, can be suspended or dissolved. Parenteral solutions are normally prepared by dissolving the compound in a carrier and sterilizing by means of a filter, before filling suitable vials or ampoules and sealing. Advantageously, adjuvants such as local anaesthetics, preservatives and buffer agents can also be dissolved in the carrier. To increase stability, the composition can be frozen after filling the vial and removing the water under vacuum. Parenteral suspensions are prepared substantially in the same manner, with the difference that the compound can be suspended in the carrier instead of dissolved, and be sterilized by exposure to ethylene oxide before suspension in the sterile carrier. Advantageously, a surfactant or a wetting agent can be included in the composition to facilitate uniform distribution of the compound of the invention.

As in common practise, the compositions are normally accompanied by written or printed instructions, for use in the treatment in question.

Consequently, another aspect of the present invention provides a pharmaceutical composition comprising a compound of formula (I), or a pharmaceutically suitable salt or solvate thereof, and a pharmaceutically acceptable carrier therefor.

The invention will now be illustrated by means of the following non-limiting examples.

### EXAMPLE 1

### 3-[4-(2,6-Dichloro-phenyl)-piperidin-1-ylmethyl]-2-phenyl-1H-indole hydrochloride

1.17 g (5.1 mmol) of 4-(2,6-dichloro-phenyl)-piperidine were dissolved in 4 mL of dioxane, 4 mL of glacial acetic acid and 0.41 mL (5.1 mmol) of a 37% aqueous solution of formaldehyde were added, followed by a solution of 893 mg (4.62 mmol) of 2-phenyl-1H-indole in 8 mL of dioxane. The reaction mixture was stirred 3 h at room temperature, then water was added followed by conc. NH₄OH up to basic pH. The reaction mixture was extracted with AcOEt, the organic phase was dried with Na₂SO₄ and the solvent removed in vacuo, yielding 2 g of crude compound. 1 g of free base was dissolved in CH₂Cl₂, the solution was brought to acidic pH with Et₂O/HCl and the solvent was removed in vacuo. The resulting solid was triturated with Et₂O, filtered and dried, yielding 1 g of the title compound as a white solid.

M.p.=169-171° C. IR (KBr, cm⁻¹)=3429, 2370, 1435. NMR (free base, 300 MHz, CDCl₃, δ ppm): 8.19 (s br, 1H); 7.91-7.81 (m, 3H); 7.49 (dd, 2H); 7.39 (d, 2H); 7.29-7.12 (m, 4H); 7.01 (dd, 1H); 3.75 (s, 2H); 3.52 (tt, 1H); 3.12 (m, 2H); 2.63 (dq, 2H); 2.17 (dq, 2H); 1.53 (m, 2H). MS (m/z): 435 (MH⁺).

Compounds described in Example 2 and Example 3 were obtained following procedure described in Example 1.

### EXAMPLE 2

### 3-[4-(2,6-Dichloro-phenyl)-piperidin-1-ylmethyl]-1H-indole hydrochloride

M.p.=189-190° C. NMR (free base, 300 MHz, CDCl₃, δ ppm): 8.10 (s br, 1H); 7.77 (d, 1H); 7.38 (d, 1H); 7.27-7.11 (m, 5H); 7.01 (dd, 1H); 3.80 (s, 2H); 3.53-3.40 (m, 1H); 3.13 (m, 2H); 2.67 (dq, 2H); 2.15 (dt, 2H); 1.64-1.49 (m, 2H). MS (m/z): 358 (M⁺.); 228; 194; 130.

### EXAMPLE 3

### 3-[4-(2,6-Dichloro-phenyl)-piperidin-1-ylmethyl]-2-methyl-1H-indole

M.p.=168-170° C. IR (KBr, cm⁻¹)=3401, 2904, 1432. NMR (300 MHz, CDCl₃, 8 ppm): 7.80 (s br, 1H); 7.69 (m, 1H); 7.31-7.19 (m, 3H); 7.15-7.07 (m, 2H); 7.01 (dd, 1H); 3.73 (s, 2H); 3.45 (tt, 1H); 3.08 (m, 2H); 2.71-2.55 (m, 2H); 2.46 (s, 3H); 2.13 (m, 2H); 1.51 (m, 2H). MS (m/z): 372 (M⁺.); 230; 228; 144; 143.

### EXAMPLE 4

### 2-(4-Chloro-phenyl)-3-[4-(2,6-dimethyl-phenyl)-piperidin-1-ylmethyl]-1H-indole

To a solution of 48 mg (0.253 mmol) of 4-(2,6-dimethyl-phenyl)-piperidine in 1 mL of MeOH:dioxane mixture (8:2 respectively), 0.021 mL (0.278 mmol) of CH₂O (37% aqueous solution) and 0.017 mL (0.304 mmol) of glacial AcOH were added at room temperature. After stirring for 20 minutes, a solution of 63 mg (0.326 mmol) of 2-(4-chloro-phenyl)-1H-indole in 2.5 mL of MeOH:dioxane mixture (8:2 respectively) was added, and the resulting mixture was stirred at room temperature overnight. The reaction mixture was poured onto SCX cartridge and eluted with 24 mL of CH₂Cl₂ and 36 mL of MeOH to remove excess starting material and then with 18 mL of 3% methanolic ammonia solution to recover the title compound. The solvent was removed in vacuo, yielding 100 mg of the title compound as a white solid.

MS (m/z): 429 (MH⁺).

Compounds of formula (I) and described in Table 1 were obtained following procedure described in Example 4.

TABLE 1

| | |
|---|---|
| | (1) |
| 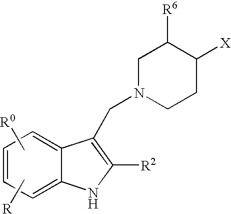 | |

| Ex. | | | | | | MS | | |
|---|---|---|---|---|---|---|---|---|
| no | R | R⁰ | R² | R⁶ | X | (m/z) | NMR (300 MHz, δ ppm) | Name |
| 5 | H | H | 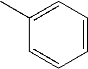 | H | 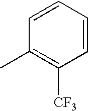 | 435 (MH⁺) | — | 2-Phenyl-3-[4-(2- trifluoromethyl- phenyl)-piperidin- 1-ylmethy]-1H- indole |
| 6 | H | H | 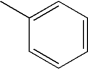 | H | 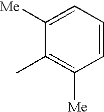 | 395 (MH⁺) | — | 3-[4-(2,6-Dimethyl- phenyl)-piperidin- 1-ylmethyl]-2- phenyl-1H-indole |
| 7 | H | H | 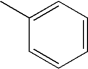 | H | 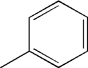 | 367 (MH⁺) | — | 2-Phenyl-3-(4- phenyl-piperidn-1- ylmethyl)-1H- indole |
| 8 | H | H | 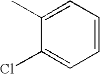 | H | 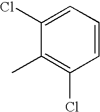 | 469 (MH⁺) | — | 2-(2-Chloro- phenyl)-3-[4-(2,6- dichloro-phenyl)- piperidin-1- ylmethyl]-1H- indole |
| 9 | H | H | 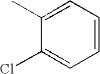 | H | 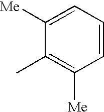 | 429 (MH⁺) | — | 2-(2-Chloro- phenyl)-3-[4-(2,6- dimethyl-phenyl)- piperidin-1- ylmethyl]-1H- indole |
| 10 | H | H | 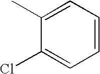 | H | 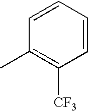 | 469 (MH⁺) | — | 2-(2-Chloro- phenyl)-3-[4-(2- trifluoromethyl- phenyl)-piperidin- 1-ylmethyl]-1H- indole |
| 11 | H | H | 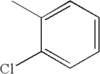 | H | 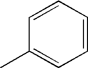 | 401 (MH⁺) | — | 2-(2-Chloro- phenyl)-3-(4- phenyl-piperidin-1- ylmethyl)-1H- indole |
| 12 | H | H | 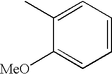 | H | 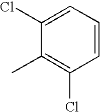 | 465 (MH⁺) | — | 3-[4-(2,6-Dichloro- phenyl)-piperidin- 1-ylmethyl]-2-(2- methoxy-phenyl)- 1H-indole |
| 13 | H | H | 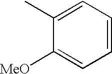 | H | 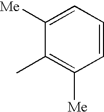 | 425 (MH⁺) | — | 3-[4-(2,6-Dimethyl- phenyl)-piperidin- 1-ylmethyl]-2-(2- methoxy-phenyl)- 1H-indole |
| 14 | H | H | 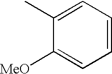 | H | 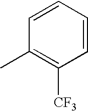 | 465 (MH⁺) | — | 2-(2-Methoxy- phenyl)-3-[4-(2- trifluoromethyl- phenyl)-piperidin- 1-ylmthyl]-1H- indole |
| 15 | H | H | 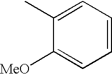 | H | 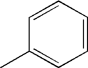 | 397 (MH⁺) | — | 2-(2-Methoxy- phenyl)-3-(4- phenyl-piperidin-1- ylmethyl)-1H- indole |
| 16 | H | H | 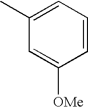 | H | 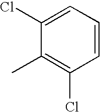 | 465 (MH⁺) | — | 3-[4-(2,6-Dichloro- phenyl)-piperidin- 1-ylmethyl]-2-(3- methoxy-phenyl)- 1H-indole |
| 17 | H | H | 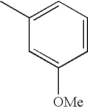 | H | 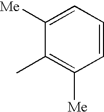 | 425 (MH⁺) | — | 3-[4-(2,6-Dimethyl- phenyl)-piperidin- 1-ylmethyl]-2-(3- methoxy-phenyl)- 1H-indole |
| 18 | H | H | 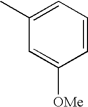 | H | 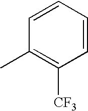 | 465 (MH⁺) | — | 2-(3-Methoxy- phenyl)-3-[4-(2- trifluoromethyl- phenyl)-piperidin- 1-ylmethyl]-1H- indole |
| 19 | H | H | 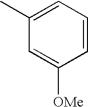 | H | 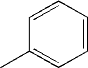 | 397 (MH⁺) | — | 2-(3-Methoxy- phenyl-3-(4- phenyl-piperidin-1- ylmethyl)-1H- indole |
| 20 | H | H | 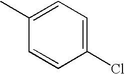 | H | 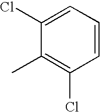 | 469 (MH⁺) | — | 2-(4-Chloro- phenyl)-3-[4-(2,6- dichloro-phenyl)- piperidin-1- ylmethyl]-1H- indole |
| 21 | H | H | 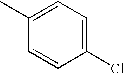 | H | 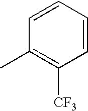 | 469 (MH⁺) | — | 2-(4-Chloro- phenyl)-3-[4-(2- trifluoromethyl- phenyl)-piperidin- 1-ylmethyl]-1H- indole |
| 22 | H | H | 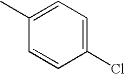 | H | 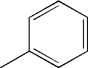 | 401 (MH⁺) | — | 2-(4-Chloro- phenyl)-3-(4- phenyl-piperidin-1- ylmethyl)-1H- indole |
| 23 | H | H | 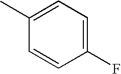 | H | 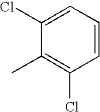 | 453 (MH⁺) | — | 3-[4-(2,6-Dichloro- phenyl)-piperidin- 1-ylmethyl]-2-(4- fluoro-phenyl)-1H- indole |
| 24 | H | H | 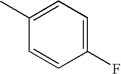 | H | 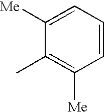 | 413 (MH⁺) | — | 3-[4-(2,6-Dimethyl- phenyl)-piperidin- 1-ylmethyl]-2-(4- fluoro-phenyl)-1H- indole |
| 25 | H | H | 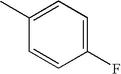 | H | 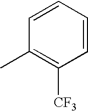 | 453 (MH⁺) | — | 2-(4-Fluoro- phenyl)-3-[4-(2- trifluoromethyl- phenyl)-piperidin- 1-ylmethyl]-1H- indole |
| 26 | H | H | 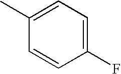 | H | 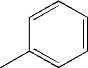 | 385 (MH⁺) | — | 2-(4-Fluoro- phenyl)-3-(4- phenyl-piperidin-1- ylmethyl)-1H- indole |
| 27 | H | H | 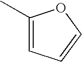 | H | 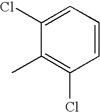 | 425 (MH⁺) | — | 3-[4-(2,6-Dichloro- phenyl)-piperidin- 1-ylmethyl]-2- furan-2-yl-1H- indole |
| 28 | H | H | 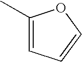 | H | 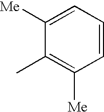 | 385 (MH⁺) | — | 3-[4-(2,6-Dimethyl- phenyl)-piperidin- 1-ylmethyl]-2- furan-2-yl-1H- indole |
| 29 | H | H | 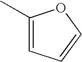 | H | 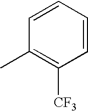 | 425 (MH⁺) | — | 2-Furan-2-yl-3-[4- (2-trifluoromethyl- phenyl)-piperidin- 1-ylmethyl]-1H- indole |
| 30 | H | H | 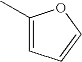 | H | 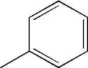 | 357 (MH⁺) | — | 2-Furan-2-yl-3-(4- phenyl-piperidin-1- ylmethyl)-1H- indole |
| 31 | H | H | 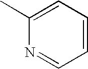 | H | 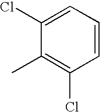 | 436 (MH⁺) | — | 3-[4-(2,6-Dichloro- phenyl)-piperidin- 1-ylmethyl]-2- pyridin-2-yl-1H- indole |
| 32 | H | H | 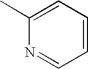 | H | 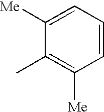 | 396 (MH⁺) | — | 3-[4-(2,6-Dimethyl- phenyl)-piperidin- 1-ylmethyl]-2- pyridin-2-yl-1H- indole |
| 33 | H | H | 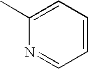 | H | 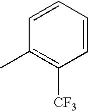 | 436 (MH⁺) | — | 2-Pyridin-2-yl-3-[4- (2-trifluoromethyl- phenyl)-piperidin- 1-ylmethyl]-1H- indole |
| 34 | H | H | 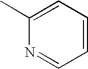 | H | 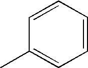 | 368 (MH⁺) | — | 3-(4-Phenyl- piperidin-1- ylmethyl)-2- pyridin-2-yl-1H- indole |
| 35 | H | H | 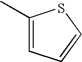 | H | 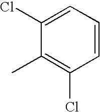 | 441 (MH⁺) | — | 3-[4-(2,6-Dichloro- phenyl)-piperidin- 1-ylmethyl]-2- thiophen-2-yl-1H- indole |
| 36 | H | H | 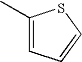 | H | 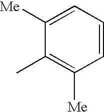 | 401 (MH⁺) | — | 3-[4-(2,6-Dimethyl- phenyl)-piperidin- 1-ylmethyl]-2- thiophen-2-yl-1H- indole |
| 37 | H | H | 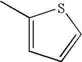 | H | 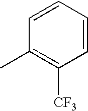 | 441 (MH⁺) | — | 2-Thiophen-2-yl-3- [4-(2- trifluoromethyl- phenyl)-piperidin- 1-ylmethyl]-1H- indole |
| 38 | H | H | 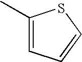 | H | 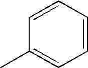 | 373 (MH⁺) | — | 3-(4-Phenyl- piperidin-1- ylmethyl)-2- thiophen-2-yl-1H- indole |
| 39 | H | H | 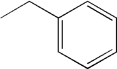 | H | 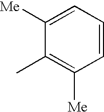 | 409 (MH⁺) | — | 2-Benzyl-3-[4-(2,6- dimethyl-phenyl)- piperidin-1- ylmethyl]-1H- indole |
| 40 | H | H | 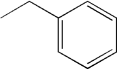 | H | 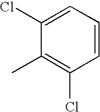 | 449 (MH⁺) | — | 2-Benzyl-3-[4-(2,6- dichloro-phenyl)- piperidin-1- ylmethyl]-1H- indole |
| 41 | H | H | 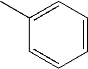 | H | 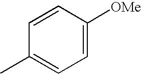 | 397 (MH⁺) | — | 3-[4-(4-Methoxy- phenyl)-piperidin- 1-ylmethyl]-2- phenyl-1H-indole |
| 42 | H | H | 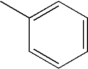 | H | 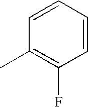 | 385 (MH⁺) | — | 3-[4-(2-Fluoro- phenyl)-piperidin- 1-ylmethyl]-2- phenyl-1H-indole |
| 43 | H | H | 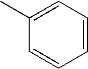 | H | 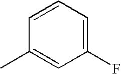 | 385 (MH⁺) | — | 3-[4-(3-Fluoro- phenyl)-piperidin- 1-ylmethyl]-2- phenyl-1H-indole |
| 44 | H | H | 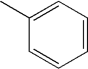 | H | 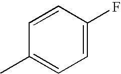 | 385 (MH⁺) | — | 3-[4-(4-Fluoro- phenyl)-piperidin- 1-ylmethyl]-2- phenyl-1H-indole |
| 45 | H | H | 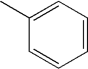 | H | 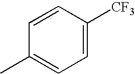 | 435 (MH⁺) | — | 2-Phenyl-3-[4-(4- trifluoromethyl- phenyl)-piperidin- 1-ylmethyl]-1H- indole |
| 46 | H | H | 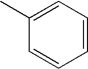 | H | 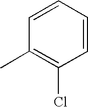 | 401 (MH⁺) | — | 3-[4-(2-Chloro- phenyl)-piperidin- 1-ylmethyl]-2- phenyl-1H-indole |
| 47 | H | H | 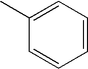 | H | 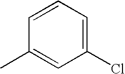 | 401 (MH⁺) | — | 3-[4-(3-Chloro- phenyl)-piperidin- 1-ylmethyl]-2- phenyl-1H-indole |
| 48 | H | H | 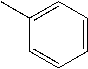 | H | 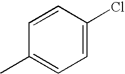 | 401 (MH⁺) | — | 3-[4-(4-Chloro- phenyl)-piperidin- 1-ylmethyl]-2- phenyl-1H-indole |
| 49 | H | H | 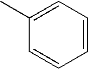 | H | 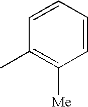 | 381 (MH⁺) | — | 2-Phenyl-3-(4-o- tolyl-piperidin-1- ylmethyl)-1H- indole |
| 50 | H | H | 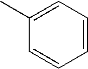 | H | 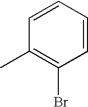 | 445 (MH⁺) | — | 3-[4-(2-Bromo- phenyl)-piperidin- 1-ylmethyl]-2- phenyl-1H-indole |
| 51 | H | H | 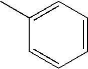 | H | 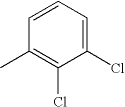 | 435 (MH⁺) | — | 3-[4-(2,3-Dichloro- phenyl)-piperidin- 1-ylmethyl]-2- phenyl-1H-indole |
| 52 | H | H | 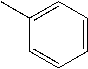 | H | 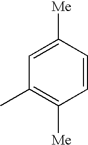 | 395 (MH⁺) | — | 3-[4-(2,5-Dimethyl- phenyl)-piperidin- 1-ylmethyl]-2- phenyl-1H-indole |
| 53 | H | H | 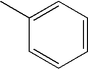 | H | 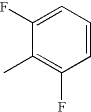 | 403 (MH⁺) | — | 3-[4-(2,6-Difluoro- phenyl)-piperidin- 1-ylmethyl]-2- phenyl-1H-indole |
| 54 | H | H | 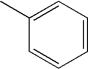 | H | 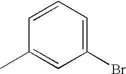 | 445 (MH⁺) | — | 3-[4-(3-Bromo- phenyl)-piperidin- 1-ylmethyl]-2- phenyl-1H-indole |
| 55 | H | H | 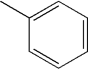 | H | 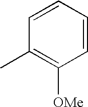 | 397 (MH⁺) | — | 3-[4-(2-Methoxy- phenyl)-piperidin- 1-ylmethyl]-2- phenyl-1H-indole |
| 56 | 5-F | H | 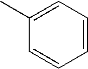 | H | 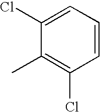 | 453 (MH⁺) | (CDCl₃-as fre base): 8.16(s br, 1H); 7.85(d, 2H); 7.55-7.45(m, 3H); 7.40(dd, 1H); 7.32-7.18 (m, 3H); 7.02(dd, 1H); 6.94(ddd, 1H); 3.70(s, 2H); 3.52(tt, 1H); 3.11 (d br, 2H); 2.66(dq, 2H); 2.19(dt, 2H); 1.53(d br, 2H) | 3-[4-(2,6-Dichloro- phenyl)-piperidin- 1-ylmethyl]-5- fluoro-2-phenyl- 1H-indole- hydrochloride |
| 57 | H | H | H | H | 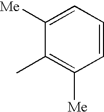 | 319 (MH⁺) | (CDCl₃): 12.32(s br, 1H); 9.36(s, 1H); 7.96 (d, 1H); 7.56(d br, 1H); 7.53(d br, 1H); 7.26 (dt, 1H); 7.21(dt, 1H); 7.02-6.95(m, 3H); 4.38 (d, 2H); 3.65(d br, 2H); 3.12(m, 1H); 2.99(dq, 2H); 2.80(dq, 2H); 2.45 (s br, 6H); 1.79(d br, 2H) | 3-[4-(2,6-Dimethyl- phenyl)-piperidin- 1-ylmethyl]-1H- indole hydrochloride |
| 58 | H | H | Me | H | 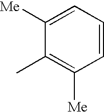 | 333 (MH⁺) | (CDCl₃): 7.90(s br, 1H); 7.68(m, 1H); 7.28(m, 1H); 7.16-7.06(m, 2H); 6.96(s, 3H); 3.74(s, 2H); 3.11(d br, 2H); 2.93(dt, 1H); 2.45(s, 3H); 2.39(s br, 6H); 2.34-2.04(m, 4H); 1.58 (d br, 2H) | 3-[4-(2,6-Dimethyl- phenyl)-piperidin- 1-ylmethyl]-2- methyl-1H-indole |
| 59 | H | H | 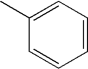 | CH₂OH | 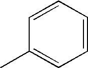 | 397 (MH⁺) | (CDCl₃): 8.24(s br, 1H); 7.76(d br, 1H); 7.65(d br, 2H); 7.50(dd, 2H); 7.40(m, 2H); 7.32-7.15 (m, 7H); 3.82 and 3.77 (ABq, 2H); 3.61-3.49(m, 2H); 3.33(dt, 1H); 3.21 (m, 1H); 2.90(dt, 1H); 2.58-2.42(m, 2H); 2.18 (dt, 1H); 1.82(m, 1H); 1.72(m, 1H) | Cis-[4-Phenyl-1- (2-phenyl-1H- indol-3-ylmethyl)- piperidin-3-yl]- methanol |
| 60 | H | H | 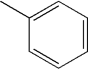 | CH₂OH | 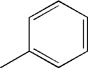 | 396 (M^{+.}) | (CDCl₃-343 K): 8.05(s br, 1H); 7.85(d, 1H); 7.80(d, 2H); 7.48(dd, 2H); 7.38(dd, 2H); 7.31- 7.12(m, 7H); 3.81(m, 2H); 3.39(m, 1H); 3.26 (m, 2H); 3.07(m, 1H); 2.36(m, 1H); 2.20-1.97 (m, 3H); 1.88-1.71(m, 2H) | Trans-[4-Phenyl-1- (2-phenyl-1H- indol-3-ylmethyl)- piperidin-3-yl]- methanol |
| 61 | 5-Cl | H | 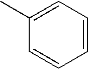 | H | 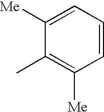 | 428 (M^{+.}) | (CDCl₃): 8.18(s br, 1H); 7.84(d, 1H); 7.83(d, 2H); 7.50(dd, 2H); 7.40 (dd, 1H); 7.30(d, 1H); 7.20(dd, 1H); 6.97(s, 3H); 3.69(s, 2H); 3.10(d br, 2H); 3.02(tt, 1H); 2.42(s br, 6H); 2.25(dq, 2H); 2.15(dt, 2H); 1.59 (d, 2H) | 5-Chloro-3-[4-(2,6- dimethyl-phenyl)- piperidin-1- ylmethyl]-2- phenyl-1H-indole |
| 62 | 5-OMe | H | 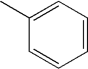 | H | 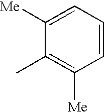 | 424 (M^{+.}) | (CDCl₃): 8.02(s br, 1H); 7.82(d, 2H); 7.49(dd, 2H); 7.37(dd, 1H); 7.35 (d, 1H); 7.28(d, 1H); 6.97(s, 3H); 6.88(dd, 1H); 3.91(s, 3H); 3.72 (s, 2H); 3.14(d br, 2H); 3.02(tt, 1H); 2.41(s br, 6H); 2.25(dq, 2H); 2.15 (dt, 2H); 1.59(d br, 2H) | 3-[4-(2,6-Dimethyl- phenyl)-piperidin- 1-ylmethyl]-5- methoxy-2-phenyl- 1H-indole |
| 63 | 5,6-(OCH₂O)— | | 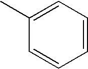 | H | 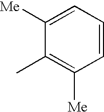 | 438 (M^{+.}) | (CDCl₃): 8.96(s br, 1H); 7.77(d br, 2H); 7.41(dd, 2H); 7.28(dd, 1H); 7.17 (s, 1H); 6.91(s, 3H); 6.83(s, 1H); 5.89(s, 2H); 3.61(s br, 2H); 3.08 (d br, 2H); 2.95(tt, 1H); 2.36(s br, 6H); 2.27- 2.02(m, 4H); 1.54(d br, 2H) | 7-[4-(2,6-Dimethyl- phenyl)-piperidin- 1-ylmethyl]-6- phenyl-5H- [1,3]dioxolo[4,5- f]indole |
| 64 | 5-OH | H | 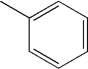 | H | 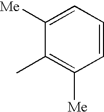 | 410 (M^{+.}) | (CDCl₃): 8.07(s br, 1H); 7.81(d, 2H); 7.48(dd, 2H); 7.37(dd, 1H); 7.25 (d, 1H); 7.23(d, 1H); 6.96(s, 3H); 6.78(dd, 1H); 3.71(s, 2H); 3.12(d br, 2H); 2.98(tt, 1H); 2.40(s br, 6H); 2.26(dq, 2H); 2.15(dt, 2H); 1.58 (d br, 2H) | 3-[4-(2,6-Dimethyl- phenyl)-piperidin- 1-ylmethyl]-1-(2- hydroxy-ethyl)-2- phenyl-1H-indol-5- ol |
| 65 | 7-Br | H | Me | H | 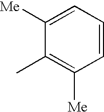 | 410 (M^{+.}) | (CDCl₃): 8.00(s br, 1H); 7.64(d, 1H); 7.27(d, 1H); 6.98(dd, 1H); 6.96 (s, 3H); 3.69(s br, 2H); 3.07(d br, 2H); 2.94(tt, 1H); 2.49(s, 3H); 2.40(s br, 6H); 2.26(dq, 2H); 2.10(dt, 2H); 1.58(d br, 2H) | 7-Bromo-3-[4-(2,6- dimethyl-phenyl)- piperidin-1- ylmethyl[-2- methyl-1H-indole |
| 66 | 5-F | H | Me | H | 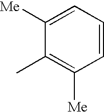 | 351 (MH⁺) | (CDCl₃): 7.81(s br, 1H); 7.34(dd, 1H); 7.17(dd, 1H); 6.96(s, 3H); 6.86 (ddd, 1H); 3.66(s br, 2H); 3.08(d br, 2H); 2.95(tt, 1H); 2.44(s, 3H); 2.40(s br, 6H); 2.24 (m, 2H); 2.09(m, 2H); 1.58(d br, 2H) | 3[4-(2,6-Dimethyl- phenyl)-piperidin- 1-ylmethyl]-5- fluoro-2-methyl- 1H-indole |
| 67 | 5-F | H | 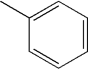 | H | 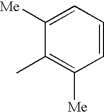 | 413 (MH⁺) | (CDCl₃: 12.29(t br, 1H); 9.87(s, 1H); 7.56(dd, 1H); 7.49-7.33(m, 5H); 7.14(dd, 1H); 7.09(ddd, 1H); 7.00-6.89(m, 3H); 4.30(d, 2H); 3.03(d br, 2H); 2.93-2.71(m, 3H); 2.45(m, 2H); 2.34(s br, 6H); 1.56(d br, 2H) | 3-[4-(2,6-Dimethyl- phenyl)-piperidin- 1-ylmethyl]-5- fluoro-2-phenyl- 1H-indole hydrochloride |

### EXAMPLE 68

### 3-[4-(2,6-Dichloro-phenyl)-piperidin-1-ylmethyl]-1H-indole-2-carboxylic acid ethyl ester

503 mg (2.66 mmol) of 4-(2,6-dichloro-phenyl)-piperidine were dissolved in 8 mL of MeOH:dioxane mixture (8:2 respectively); 0.169 mL (2.25 mmol) of CH₂O (37% aqueous solution) and 0.141 mL (2.45 mmol) of glacial AcOH were added at room temperature. After stirring for 30 minutes, a solution of 503 mg (2.66 mmol) of 1H-indole-2-carboxylic acid ethyl ester in 20 mL of MeOH:dioxane mixture (8:2 respectively) was added. The reaction mixture was heated to 50° C. for 5 h, then 2 mL of AcOH were added and heating was continued for 13 h. The volatiles were removed in vacuo, the residue was taken up in H₂O/AcOEt and concd. NH₄OH was added up to basic pH. After extraction with AcOEt the organic phase was collected, dried with Na₂SO₄ and the solvent removed in vacuo. The crude product was purified by chromatography, eluting with a mixture CH₂Cl₂/MeOH/concd. NH₄OH 100:1:0.1 respectively, yielding 410 mg of the title compound.

NMR (300 MHz, CDCl₃, δ ppm): 8.79 (s br, 1H); 8.04 (d, 1H); 7.41-7.29 (m, 2H); 7.29-7.11 (m, 3H); 7.01 (dd, 1H); 4.43 (q, 2H); 4.18 (s br, 2H); 3.48 (tt, 1H); 3.11 (m, 2H); 2.67 (m, 2H); 2.21 (m, 2H); 1.50 (m, 2H); 1.44 (t, 3H). MS (m/z): 431 (MH⁺).

Compounds described in Examples 69, 70 and 71 were obtained following procedure described in Example 68.

### EXAMPLE 69

### 3-[4-(2,6-Dimethyl-phenyl)-piperidin-1-ylmethyl]-2-phenyl-1H-indole-6-carbonitrile hydrochloride

NMR (300 MHz, CDCl₃, δ ppm): 12.37 (s br, 1H); 10.36 (s, 1H); 7.81 (d, 1H); 7.76 (s, 1H); 7.60 (m, 2H); 7.52-7.43 (m, 3H); 7.40 (dd, 1H); 7.02-6.90 (m, 3H); 4.49 (d, 2H); 3.39 (d br, 2H); 3.10-2.88 (m, 3H); 2.58 (m, 2H); 2.39 (s br, 6H); 1.69 (d br, 2H). MS (m/z): 420 (MH⁺).

### EXAMPLE 70

### 3-[4-(2,6-Dimethyl-phenyl)-piperidin-1-ylmethyl]-1,2-diphenyl-1H-indole hydrochloride

NMR (300 MHz, CDCl₃, δ ppm): 12.66 (s br, 1H); 7.80 (m, 1H); 7.42-7.27 (m, 9H); 7.24-7.15 (m, 4H); 7.00-6.90 (m, 3H); 6.45 (d, 2H); 3.15 (d br, 2H); 3.02-2.78 (m, 3H); 2.60 (m, 2H); 2.38 (s br, 6H); 1.60 (d br, 2H). MS (m/z): 471 (MH⁺).

### EXAMPLE 71

### 3-[4-(2,6-Dimethyl-phenyl)-piperidin-t-ylmethyl]-5-fluoro-1H-indole-2-carboxylic acid amide trifluoroacetate

NMR (300 MHz, CDCl₃, δ ppm): 13.13 (s, 1H); 11.79 (s br, 1H); 9.90 (s br, 1H); 7.60 (dd, 1H); 7.16-6.99 (m, 6H); 4.43 (s br, 2H); 3.71 (m, 2H); 3.30 (m, 1H); 3.00 (m, 2H); 2.55 (m, 2H); 2.41 (s, 6H); 1.99 (m, 2H). MS (m/z): 380 (MH⁺).

### EXAMPLE 72

### 3-{1-[4-(2,6-Dimethyl-phenyl)-piperidin-1-yl]-ethyl}-1H-indole hydrochloride

565 mg (4.82 mmol) of indole were dissolved in 3 mL of glacial AcOH; after cooling to 0° C., a solution of 913 mg (4.82 mmol) of 4-(2,6-dimethyl-phenyl)-piperidine in 1.5 mL of toluene was added, followed by a solution of 0.3 mL (5.3 mmol) of acetaldehyde in 1 mL of toluene. The reaction mixture was kept 96 h at 5° C., then it was poured onto ice/water, basified with NaOH solution and extracted with AcOEt. The organic phase was collected, dried with Na₂SO₄ and the solvent removed in vacuo. The resulting crude product was purified by chromatography, eluting with a mixture CH₂Cl₂/MeOH/concd. NH₄OH 100:1:0.1 respectively, yielding 814 mg of the title compound as a free base.

100 mg were dissolved in CH₂Cl₂, the solution was brought to acidic pH with Et₂O/HCl and the solvent was removed in vacuo. The resulting solid was crystallized from acetone, yielding 71 mg of the title compound.

NMR (300 MHz, CDCl₃, δ ppm): 12.14 (s br, 1H); 10.13 (s, 1H); 7.57 (d, 1H); 7.55 (d, 1H); 7.46 (d, 1H); 7.22 (dd, 1H); 7.16 (dd, 1H); 6.99-6.87 (m, 3H); 4.70 (m, 1H); 3.71 (m, 1H); 3.62 (m, 1H); 3.14 (m, 1H); 2.73-2.55 (m, 2H); 2.12 (s, 6H); 2.01 (d, 3H) 1.69 (m, 2H). MS (m/z): 333 (MH⁺).

### EXAMPLE 73

### {3-[4-(2,6-Dichloro-phenyl)-piperidin-1-ylmethyl]-1H-indol-2-yl}-methanol hydrochloride

100 mg (0.232 mmol) of 3-[4-(2,6-dichloro-phenyl)-piperidin-1-ylmethyl]-1H-indole-2-carboxylic acid ethyl ester (compound described in Example 68) were dissolved in 3 mL of dry THF under a nitrogen atmosphere. The resulting solution was cooled to −20° C. and 1.16 mL of a 1M solution of DIBAL in hexane were added dropwise. The reaction mixture was allowed to warm to −5° C. during 1 h, then it was quenched with a saturated solution of Rochelle's salt and stirred vigorously while allowed to warm to room temperature. Water was added and the reaction mixture was extracted with AcOEt; the organic phase was collected, washed with brine, dried over Na₂SO₄ and the solvent removed in vacuo. The crude product was purified by chromatography, eluting with a mixture CH₂Cl₂/MeOH/concd. NH₄OH 100:2:0.2 respectively, yielding 68 mg of the title compound as a free base. It was dissolved in CH₂Cl₂, the solution was brought to acidic pH with Et₂O/HCl and the solvent was removed in vacuo. The resulting solid was triturated with Et₂O, filtered and dried, yielding 62 mg of the title compound.

NMR (300 MHz, DMSO—343 K, δ ppm): 11.25 (s br, 1H); 7.75 (d, 1H); 7.42 (m, 3H); 7.27 (dd, 1H); 7.14 (dt, 1H); 7.09 (dt, 1H); 4.81 (s, 2H); 4.46 (s br, 2H); 3.72 (tt, 1H); 3.54 (d br, 2H); 3.11 (m, 2H); 2.81 (m, 2H); 1.77 (d br, 2H). MS (m/z): 387 (M⁺.), 230, 194.

### EXAMPLE 74

### 1-Benzyl-3-[4-(2,6-dichloro-phenyl)-piperidin-1-ylmethyl]-2-phenyl-1H-indole hydrochloride

Under a nitrogen atmosphere, 13.2 mg (0.331 mmol) of NaH (60% dispersion in mineral oil) were suspended in 0.75 mL of dry DMF. After cooling to 0° C., a solution of 120 mg (0.276 mmol) of 3-[4-(2,6-dichloro-phenyl)-piperidin-1-ylmethyl]-2-phenyl-1H-indole in 0.75 mL of dry DMF was added dropwise. The reaction mixture was stirred 30 min, then 0.036 mL (0.304 mmol) of benzyl bromide were added dropwise. The reaction mixture was allowed to warm to room temperature and stirred 2 h, then it was cooled to 0° C., water was added, followed by conc. NH₄OH and the resulting mixture was extracted with Et₂O. The organic layer was dried with Na₂SO₄ and the solvent was removed in vacuo, yielding 122 mg of crude product which was dissolved in CH₂Cl₂, the solution was brought to acidic pH with Et₂O/HCl and the solvent was removed in vacuo. The resulting solid was triturated with hot acetone, filtered and dried, yielding 53 mg of the title compound as a white solid.

M.p.=209-210° C. NMR (free base, 300 MHz, CDCl₃, δ ppm): 7.94 (m, 1H); 7.45-7.35 (m, 5H); 7.28-7.14 (m, 9H); 7.00 (dd, 1H); 6.95 (m, 1H); 5.23 (s, 2H); 3.68 (s, 2H); 3.43 (tt, 1H); 3.02 (m, 2H); 2.59 (dq, 2H); 2.04 (dt, 2H); 1.47 (m, 2H). MS (m/z): 525 (MH⁺).

Compounds described in Examples 75-86 were obtained following procedure described in Example 74.

### EXAMPLE 75

### 3-[4-(2,6-Dichloro-phenyl)-piperidin-1-ylmethyl]-2-phenyl-1-propyl-1H-indole hydrochloride

M.p.=150-152° C. NMR (free base, 300 MHz, CDCl₃, δ ppm): 7.91 (d, 1H); 7.51-7.39 (m, 5H); 7.36 (d, 1H); 7.27-7.19 (m, 3H); 7.15 (dd, 1H); 7.00 (t, 1H); 3.98 (dd, 2H); 3.63 (s, 2H); 3.41 (tt, 1H); 2.98 (m, 2H); 2.56 (dq, 2H); 2.00 (dt, 2H); 1.66 (m, 2H); 1.44 (m, 2H); 0.74 (t, 3H). MS (m/z): 476 (M⁺.); 248; 206; 178.

### EXAMPLE 76

### 3-[4-(2,6-Dichloro-phenyl)-piperidin-1-ylmethyl]-1-methyl-2-phenyl-1H-indole hydrochloride

M.p.=179-184° C. (dec.). NMR (free base, 300 MHz, CDCl₃, δ ppm): 7.90 (d, 1H); 7.55-7.40 (m, 5H); 7.35 (d, 1H); 7.29-7.15 (m, 4H); 7.00 (t, 1H); 3.65 (s, 2H); 3.63 (s, 3H); 3.43 (tt, 1H); 3.01 (m, 2H); 2.58 (dq, 2H); 2.05 (dt, 2H); 1.46 (m, 2H). MS (m/z): 448 (M⁺.); 220; 204.

### EXAMPLE 77

### 1-Benzyl-3-[4-(2,6-dichloro-phenyl)-piperidin-1-ylmethyl]-5-fluoro-2-phenyl-1H-indole hydrochloride

NMR (300 MHz, CDCl₃, free base, δ ppm): 7.61 (d, 1H); 7.42 (m, 6H); 7.32-7.18 (m, 4H); 7.08 (m, 1H); 7.00 (dd, 1H); 6.90 (m, 3H); 5.21 (s, 2H); 3.63 (s 2H); 3.44 (tt, 1H); 3.01 (d br, 2H); 2.61 (m, 2H); 2.03 (m, 2H); 1.50 (d br, 2H). MS (m/z): 543 (MH⁺), 314.

### EXAMPLE 78

### 1-Benzyl-3-[4-(2,6-dichloro-phenyl)-piperidin-1-ylmethyl]-2-methyl-1H-indole hydrochloride

NMR (300 MHz, CDCl₃, δ ppm): 12.60 (s br, 1H); 7.60 (d, 1H); 7.40-7.20 (m, 8H); 7.09 (dd, 1H); 6.99 (m, 2H); 4.50 (d, 2H); 3.65 (d br, 2H); 3.57-3.34 (m, 3H); 3.37 (s, 2H); 2.85 (dq, 2H); 2.56 (s, 3H); 1.78 (d, br, 2H). MS (m/z): 462 (M⁺.).

### EXAMPLE 79

### 1-Benzyl-3-[4-(2,6-dichloro-phenyl)-piperidin-1-ylmethyl]-1H-indole hydrochloride

NMR (300 MHz, CDCl₃+D₂O+Na₂CO₃, δ ppm): 7.77 (dd, 1H); 7.33-7.07 (m, 11H); 7.01 (dd, 1H); 5.31 (s, 2H); 3.80(s, 2H); 3.46 (tt, 1H); 3.12 (d br, 2H); 2.66 (dq, 2H); 2.14 (dt, 2H); 1.53 (d br, 2H). MS (m/z): 449 (MH⁺), 220.

### EXAMPLE 80

### 1-Benzyl-3-[4-(2,6-dimethyl-phenyl)-piperidin-1-ylmethyl]-2-methyl-1H-indole hydrochloride

NMR (300 MHz, CDCl₃, δ ppm): 10.12 (s br, 1H); 7.83 (m, 1H); 7.45 (m, 1H); 7.34-7.21 (m, 3H); 7.13 (m, 2H); 7.03 (d, 2H); 6.95 (s, 3H); 5.49 (s, 2H); 4.48 (d, 2H); 3.54 (d br, 2H); 3.27-3.09 (m, 3H); 2.56-2.47 (m, 5H); 2.37 (s, 6H); 1.72 (d br, 2H). MS (m/z):423 (MH⁺).

### EXAMPLE 81

### 1-Benzyl-3-[4-(2,6-dimethyl-phenyl)-piperidin-1-ylmethyl]-2-phenyl-1H-indole hydrochloride

NMR (300 MHz, DMSO, δ ppm): 10.16 (s br, 1H); 8.03 (m, 1H); 7.60-7.50 (m, 5H); 7.48 (m, 1H); 7.28-7.14 (m, 5H); 6.97-6.82 (m, 5H); 5.34 (s, 2H); 4.43 (d, 2H); 3.28 (d br, 2H); 3.08 (tt, 1H); 2.84 (m, 2H); 2.41 (m, 2H); 2.31 (s br, 6H); 1.58 (d br, 2H). MS (m/z):484 (M⁺.), 296, 204.

### EXAMPLE 82

### 1-Benzyl-3-[4-(2,6-dimethyl-phenyl)-piperidin-1-ylmethyl]-1H-indole hydrochloride

NMR (300 MHz, CDCl₃, δ ppm): 12.55 (s br, 1H); 7.87 (s, 1H); 7.58 (m, 1H); 7.37-7.10 (m, 8H); 6.99 (m, 3H); 5.38 (s, 2H); 4.39 (d, 2H); 3.63 (d br, 2H); 3.16-2.92 (m, 3H); 2.77 (m, 2H); 2.44 (s br, 6H); 1.78 (d br, 2H). MS (m/z):408 (M⁺.), 220.

### EXAMPLE 83

### 1-Benzyl-5-chloro-3-[4-(2,6-dimethyl-phenyl)-piperidin-1-ylmethyl]-2-phenyl-1H-indole hydrochloride

NMR (300 MHz, DMSO, δ ppm): 9.96 (s br, 1H); 8.17 (d, 1H); 7.59-7.50 (m, 6H); 7.24 (dd, 1H); 7.22-7.16 (m, 3H); 6.98-6.88 (m, 3H); 6.84 (m, 2H); 5.34 (s, 2H); 4.42 (d, 2H); 3.32 (d br, 2H); 3.09 (tt, 1H); 2.80 (m, 2H); 2.41 (m, 2H); 2.31 (s br, 6H); 1.59 (d br, 2H). MS (m/z):518 (M⁺.), 330, 91.

### EXAMPLE 84

### 1-Benzyl-3-[4-(2,6-dimethyl-phenyl)-piperidin-1-ylmethyl]-5-methoxy-2-phenyl-1H-indole hydrochloride

NMR (300 MHz, DMSO, δ ppm): 10.25 (s br, 1H); 7.60 (m, 1H); 7.58-7.45 (m, 5H); 7.35 (d, 1H); 7.24-7.13 (m, 3H); 6.98-6.81 (m, 6H); 5.30 (s, 2H); 4.40 (d, 2H); 3.86 (s, 3H); 3.28 (d br, 2H); 3.07 (tt, 1H); 2.79 (m, 2H); 2.43 (m, 2H); 2.31 (s br, 6H); 1.58 (d br, 2H). MS (m/z):514 (M⁺.), 326, 91.

### EXAMPLE 85

### 5-Benzyl-7-[4-(2,6-dimethyl-phenyl)-piperidin-1-ylmethyl]-6-phenyl-5H-[1,3]dioxolo[4,5-q]indole hydrochloride

NMR (300 MHz, DMSO, δ ppm): 9.93 (s br, 1H); 7.57 (s, 1H); 7.56-7.45 (m, 5H); 7.26-7.13 (m, 3H); 7.08 (s, 1H); 6.99-6.88 (m, 3H); 6.84 (m, 2H); 5.99 (s, 2H); 5.27 (s, 2H); 4.26 (d, 2H); 3.27 (d br, 2H); 3.07 (tt, 1H); 2.78 (dt, 2H); 2.37 (m, 2H); 2.31 (s br, 6H); 1.58 (d br, 2H). MS (m/z):528 (M⁺.), 340, 249, 91.

### EXAMPLE 86

### {3-[4-(2,6-Dichloro-phenyl)-piperidin-1-ylmethyl]-5-fluoro-2-phenyl-indol-1-yl}-acetic acid methyl ester hydrochloride

NMR (300 MHz, CDCl₃, δ ppm): 12.68 (s br, 1H); 7.58-7.51 (m, 4H); 7.43-7.34 (m, 3H); 7.24 (m, 2H); 7.11 (dt, 1H); 7.04 (dd, 1H); 4.70 (s, 2H); 4.46 (d, 2H); 3.75 (s, 3H); 3.46-3.20 (m, 3H); 3.12 (m. 2H); 2.61 (m, 2H); 1.59 (m, 2H). MS (m/z):525 (MH⁺).

### EXAMPLE 87

### 3-(4-(2,6-Dichloro-phenyl)piperidin-1-ylmethyl)-1-(2-hydroxyethyl)-2-phenyl-1H-indole hydrochloride

Under a nitrogen atmosphere, 25 mg (0.62 mmol) of NaH (60% dispersion in mineral oil) were suspended in 0.75 mL of dry DMF. After cooling to 0° C., a solution of 150 mg (0.344 mmol) of 3-[4-(2,6-dichloro-phenyl)-piperidin-1-ylmethyl]-2-phenyl-1H-indole in 0.75 mL of dry DMF was added dropwise. The reaction mixture was stirred 30 min, then 0.093 mL (0.62 mmol) of 2-(2-bromo-ethoxy)-tetrahydro-pyran were added dropwise. The reaction mixture was allowed to warm to room temperature and stirred 2 h, then it was cooled to 0° C., water was added, followed by conc. NH₄OH and the resulting mixture was extracted with Et₂O. The organic layer was dried with Na₂SO₄ and the solvent was removed in vacuo, yielding 201 mg of crude product, which was dissolved in a mixture of 2 mL of dioxane and 3 mL of 1N HCl and stirred 1 h at room temperature. Conc. NH₄OH was added up to basic pH, then the reaction mixture was extracted with Et₂O, the organic phase was dried and the solvent removed in vacuo, yielding 130 mg of crude compound was dissolved in CH₂Cl₂, the solution was brought to acidic pH with Et₂O/HCl and the solvent was removed in vacuo. The resulting solid was crystallised from acetone, filtered and dried, yielding 44 mg of the title compound as a white solid.

M.p.=205-206° C. NMR (free base, 300 MHz, CDCl₃, δ ppm): 7.91 (d, 1H); 7.50-7.41 (m, 6H); 7.29-7.14 (m, 4H); 7.00 (dd, 1H); 4.21 (t, 2H); 3.79 (m, 2H); 3.62 (s, 2H); 3.41 (tt, 1H); 2.98 (m, 2H); 2.56 (dq, 2H); 2.01 (dt, 2H); 1.45 (m, 3H). MS (m/z): 478 (M⁺.); 250.

Compounds described in Examples 88-98 were obtained following procedure described in Example 87.

### EXAMPLE 88

### 2-{3-[4-(2,6-Dichloro-phenyl)-piperidin-1-ylmethyl]-5-fluoro-2-phenyl-indol-1-yl}-ethanol hydrochloride

NMR (300 MHz, CDCl₃, free base, δ ppm): 7.60 (dd, 1H); 7.52-7.38 (m, 5H); 7.34 (dd, 1H); 7.22 (m, 2H); 7.03-6.92 (m, 2H); 4.19 (t, 2H); 3.79 (m, 2H); 3.58 (s, 2H); 3.41 (tt, 1H); 2.97 (d br, 2H); 2.68-2.49 (m, 3H); 2.00 (dt, 2H); 1.46 (d br, 2H). MS (m/z): 497 (MH⁺).

### EXAMPLE 89

### 2-{3-[4-(2,6-Dichloro-phenyl)-piperidin-1-ylmethyl]-indol-1-yl}-ethanol

NMR (300 MHz, CDCl₃, δ ppm): 7.76 (d, 1H); 7.36 (d, 1H); 7.24 (d, 2H); 7.23 (dt, 1H); 7.18 (s, 1H); 7.14 (dt, 1H); 7.02 (dd, 2H); 4.27 (dd, 2H); 3.98 (dd, 2H); 3.80 (s, 2H); 3.48 (tt, 1H); 3.15 (d br, 2H); 2.68 (dq, 2H); 2.18 (dt, 2H); 1.55 (d br, 2H). MS (m/z): 403 (MH⁺), 174.

### EXAMPLE 90

### 2-{3-[4-(2,6-Dimethyl-phenyl)-piperidin-1-ylmethyl]-indol-1-yl}-ethanol

NMR (300 MHz, CDCl₁ 3, ppm): 7.75 (d, 1H); 7.37 (d, 1H); 7.26 (s, 1H); 7.24 (dt, 1H); 7.15 (dt, 1H); 6.96 (s, 3H); 4.29 (dd, 2H); 3.99 (dd, 2H); 3.82 (s br, 2H); 3.18 (m, 2H); 2.97 (tt, 1H); 2.48-2.11 (m, 10H); 1.62 (d br, 2H). MS (m/z): 363 (MH⁺).

### EXAMPLE 91

### 2-{3-[4-(2,6-Dimethyl-phenyl)-piperidin-1-ylmethyl]-2-methyl-indol-1-yl}-ethanol

NMR (300 MHz, CDCl₃, δ ppm): 7.69 (d, 1H); 7.31 (d, 1H); 7.17 (dt, 1H); 7.12 (dt, 1H); 6.96 (s, 3H); 4.27 (t, 2H); 3.92 (t, 2H); 3.73 (s, 2H); 3.10 (d br, 2H); 2.95 (tt, 1H); 2.48 (s, 3H); 2.40 (s br, 6H); 2.27 (m, 2H); 2.15 (m, 2H); 1.76 (s br, 1H); 1.58 (d br, 2H). MS (m/z): 377 (MH⁺).

### EXAMPLE 92

### 2-{3-[4-(2,6-Dichloro-phenyl)-piperidin-1-ylmethyl]-2-methyl-indol-1-yl}-ethanol hydrochloride

NMR (300 MHz, CDCl₃, δ ppm): 7.69 (d, 1H); 7.30 (d, 1H); 7.24 (d, 2H); 7.16 (dt, 1H); 7.11 (dt, 1H); 7.01 (dd, 1H); 4.28 (t, 2H); 3.95 (t, 2H); 3.74 (s, 2H); 3.46 (tt, 1H); 3.10 (d br, 2H); 2.63 (m, 2H); 2.48 (s, 3H); 2.16 (m, 2H); 1.53 (d br, 2H). MS (m/z): 417 (MH⁺).

### EXAMPLE 93

### 2-{3-[4-(2,6-Dimethyl-phenyl)-piperidin-1-ylmethyl]-2-phenyl-indol-1-yl}-ethanol hydrochloride

NMR (300 MHz, DMSO, δ ppm): 9.76 (s br, 1H); 7.95 (d, 1H); 7.67-7.52 (m, 6H); 7.30 (dd, 1H); 7.22 (dd, 1H); 6.97-6.87 (m, 3H); 4.83 (s br, 1H); 4.37 (d, 2H); 4.10 (t, 2H); 3.54 (t br, 2H); 3.28 (d br, 2H); 3.06 (tt, 1H); 2.81 (m, 2H); 2.38 (m, 2H); 2.30 (s br, 6H); 1.58 (d br, 2H). MS (m/z): 438 (M⁺.), 250, 218, 205.

### EXAMPLE 94

### 3-{3-[4-(2,6-Dimethyl-phenyl)-piperidin-1-ylmethyl]-2-phenyl-indol-1-yl}-propan-1-ol hydrochloride

NMR (300 MHz, CDCl₃, δ ppm): 12.51 (s br, 1H); 7.72 (d, 1H); 7.62-7.52 (m, 4H); 7.43-7.28 (m, 4H); 7.01-6.90 (m, 3H); 4.47 (d, 2H); 4.26 (dd, 2H); 3.46 (dd, 2H); 3.15 (d br, 2H); 3.03-2.76 (m, 3H); 2.59 (dt, 2H); 2.36 (s br, 6H); 1.84 (m, 2H); 1.61 (d br, 2H). MS (m/z): 452 (M⁺.), 264.

### EXAMPLE 95

### 2-{3-[4-(2,6-Dimethyl-phenyl)-piperidin-1-ylmethyl]-5-methoxy-2-phenyl-indol-1-yl}-ethanol hydrochloride

NMR (300 MHz, DMSO δ ppm): 9.77 (s br, 1H); 7.66-7.45 (m, 7H); 6.98-6.84 (m, 4H); 4.80 (t, 1H); 4.33 (s br, 2H); 4.06 (t, 2H); 3.86 (s, 3H); 3.51 (dt, 2H); 3.28 (m, 2H); 3.05 (m, 1H); 2.75 (m, 2H); 2.46-2.17 (m, 8H); 1.58 (d br, 2H). MS (m/z): 468 (M⁺.), 280, 250, 204.

### EXAMPLE 96

### 2-{5-Chloro-3-[4-(2,6-dimethyl-phenyl)-piperidin-1-ylmethyl]-2-phenyl-indol-1-yl}-ethanol hydrochloride

NMR (300 MHz, DMSO, δ ppm): 9.94 (s br, 1H); 8.12 (d, 1H); 7.68 (d, 1H); 7.64-7.54 (m, 5H); 7.29 (dd, 1H); 6.96-6.88 (m, 3H); 4.85 (s br, 1H); 4.35 (d, 2H); 4.10 (t, 2H); 3.52 (t, 2H); 3.29 (d br, 2H); 3.06 (t br, 1H); 2.74 (m, 2H); 2.39 (m, 2H); 2.30 (s br, 6H); 1.58 (d br, 2H). MS (m/z): 472 (M⁺.), 442, 284, 249.

### EXAMPLE 97

### 2{-[4-(2,6-Dimethyl-phenyl)-piperidin-1-ylmethyl]-6-phenyl-[1,3]dioxolo[4,5-f]indol-5-yl}-ethanol hydrochloride

NMR (300 MHz, DMSO, δ ppm): 9.75 (s br, 1H); 7.62-7.45 (m, 6H); 7.24 (s, 1H); 6.98-6.87 (m, 3H); 6.01 (s, 2H); 4.79 (t, 1H); 4.28 (s br, 2H); 4.02 (t, 2H); 3.49 (dt, 2H); 3.24 (d br; 2H); 3.05 (tt, 1H); 2.75 (m, 2H); 2.45-2.21 (m, 8H); 1.57 (d br, 2H). MS (m/z): 483 (MH⁺).

### EXAMPLE 98

### 2-{3-[4-(2,6-Dimethyl-phenyl)-piperidin-1-ylmethyl]-5-fluoro-2-methyl-indol-1-yl}-ethanol hydrochloride

NMR (300 MHz, CDCl₃-D₂O, δ ppm): 7.28 (m, 1H); 7.15 (dd, 1H); 6.98-6.87 (m, 4H); 4.29 (s br, 2H); 4.24 (t, 2H); 3.86 (t, 2H); 3.62 (d br, 2H); 3.09-2.89 (m, 3H); 2.80 (m, 2H); 2.62 (s br, 3H); 2.40 (s br, 6H); 1.73 (d br, 2H). MS (m/z): 395 (MH⁺).

### EXAMPLE 99

### 1-(4-tert-Butyl-benzyl)-3-[4-(2,6-dichloro-phenyl)-piperidin-1-ylmethyl]-2-phenyl-1H-indole trifluoroacetate

To a solution of 70 mg (0.16 mmol) of 3-[4-(2,6-dichloro-phenyl)-piperidin-1-ylmethyl]-2-phenyl-1H-indole in 0.5 mL of dry DMF, 15 mg (0.37 mmol) of NaH (60% dispersion in mineral oil) were added. After stirring for 15 min at room temperature under an argon atmosphere, 0.1 mL of a 2M solution of 4-tert-butyl-benzylbromide in DMF were added; after stirring for 75 min a second addition of 0.1 mL of a 2M solution of 4-tert-butyl-benzylbromide in DMF was done and the resulting mixture was stirred at room temperature overnight. The reaction was quenched with few drops of water, poured onto Chem-elute cartridge to retain water and eluted with AcOEt; the resulting solution was concentrated and then poured onto SCX cartridge and eluted with MeOH to eliminate non-basic impurities and then with 3% methanolic ammonia solution to recover the title compound. The solvent was removed in vacuo and the resulting crude product was purified by preparative HPLC on a Symmetry C18 column, by gradient elution with a solvent system water/TFA 99.9:0.1 respectively (A) and CH₃CN/TFA 99.9:0.1 respectively (B) with the following gradient: 25% B (1 min); 25% B→95% B (8 min); 95% B→25% B (1 min), yielding 27 mg of the title compound.

MS (m/z): 581 (MH⁺).

Compounds of formula (2) and described in Table 2 were obtained following procedure described in Example 99.

TABLE 2

| | |
|---|---|
| | (2) |
| 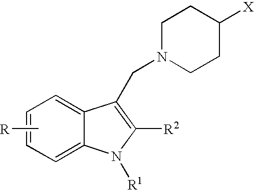 | |

| | | | | | MS | |
|---|---|---|---|---|---|---|
| Ex. no | R | R¹ | R² | X | (m/z) | Name |
| 100 | H | CH₂CH₂CHMe₂ | 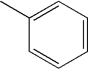 | 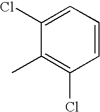 | 505 (MH⁺) | 3-[4-(2,6-Dichloro- phenyl)-piperidin-1- ylmethyl]-1-(3- methyl-butyl)-2- phenyl-1H-indole |
| 101 | H | Cyclopropyl- methyl | 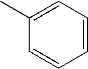 | 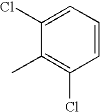 | 489 (MH⁺) | 1- Cyclopropylmethyl- 3-[4-(2,6-dichloro- phenyl)-piperidin-1- ylmethyl]-2-phenyl- 1H-indole |
| 102 | H | 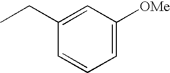 | 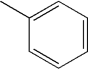 | 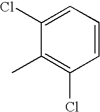 | 555 (MH⁺) | 3-[4-(2,6-Dichloro- phenyl)-piperidin-1- ylmethyl]-1-(3- methoxy-benzyl)-2- phenyl-1H-indole |
| 103 | H | 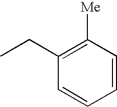 | 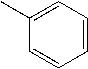 | 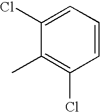 | 539 (MH⁺) | 3-[4-(2,6-Dichloro- phenyl)-piperidin-1- ylmethyl]-1-(2- methyl-benzyl)-2- phenyl-1H-indole |
| 104 | H | 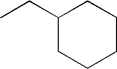 | 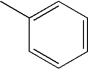 | 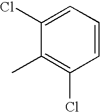 | 531 (MH⁺) | 1-Cyclohexylmethyl- 3-[4-(2,6-dichloro- phenyl)-piperidin-1- ylmethyl]-2-phenyl- 1H-indole |
| 105 | H | 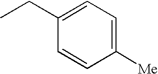 | 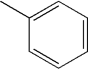 | 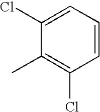 | 539 (MH⁺) | 3-[4-(2,6-Dichloro- phenyl)-piperidin-1- ylmethyl]-1-(4- methyl-benzyl)-2- phenyl-1H-indole |
| 106 | H | 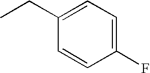 | 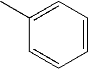 | 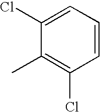 | 543 (MH⁺) | 3-[4-(2,6-Dichloro- phenyl)-piperidin-1- ylmethyl]-1-(4- fluoro-benzyl)-2- phenyl-1H-indole |
| 107 | H | 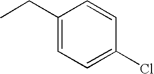 | 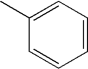 | 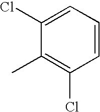 | 559 (MH⁺) | 1-(3-Chloro-benzyl)- 3-[4-(2,6-dichloro- phenyl)-piperidin-1- ylmethyl]-2-phenyl- 1H-indole trifluoroacetate |
| 108 | H | 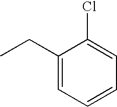 | 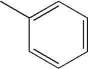 | 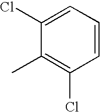 | 559 (MH⁺) | 1-(2-Chloro-benzyl)- 3-[4-(2,6-dichloro- phenyl)-piperidin-1- ylmethyl]-2-phenyl- 1H-indole |
| 109 | H | 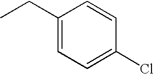 | 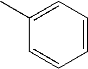 | 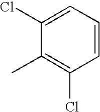 | 559 (MH⁺) | 1-(4-Chloro-benzyl)- 3-[4-(2,6-dichloro- phenyl)-piperidin-1- ylmethyl]-2-phenyl- 1H-indole |
| 110 | H | Allyl | 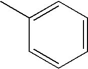 | 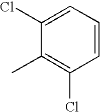 | 475 (MH⁺) | 1-Allyl-3-[4-(2,6- dichloro-phenyl)- piperidin-1- ylmethyl]-2-phenyl- 1H-indole |
| 111 | H | Propargyl | 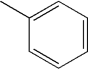 | 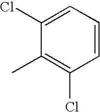 | 473 (MH⁺) | 3-[4-(2,6-Dichloro- phenyl)-piperidin-1- ylmethyl]-2-phenyl- 1-prop-2-ynyl-1H- indole |
| 112 | H | 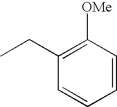 | 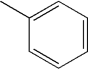 | 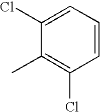 | 555 (MH⁺) | 3-[4-(2,6-Dichloro- phenyl)-piperidin-1- ylmethyl]-1-(2- methoxy-benzyl)-2- phenyl-1H-indole trifluoroacetate |
| 113 | H | 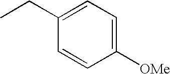 | 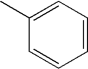 | 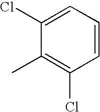 | 555 (MH⁺) | 3-[4-(2,6-Dichloro- phenyl)-piperidin-1- ylmethyl]-1-(4- methoxy-benzyl)-2- phenyl-1H-indole trifluoroacetate |
| 114 | H | 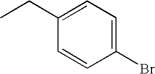 | 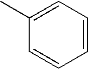 | 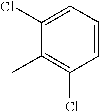 | 603 (MH⁺) | 1-(4-Bromo-benzyl)- 3-[4-(2,6-dichloro- phenyl)-piperidin-1- ylmethyl]-2-phenyl- 1H-indole trifluoroacetate |
| 115 | H | 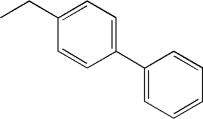 | 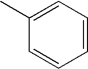 | 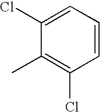 | 601 (MH⁺) | 1-Biphenyl-4- ylmethyl-3-[4-(2,6- dichloro-phenyl)- piperidin-1- ylmethyl]-2-phenyl- 1H-indole trifluoroacetate |
| 116 | H | 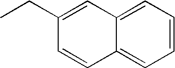 | 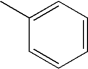 | 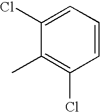 | 575 (MH⁺) | 3-[4-(2,6-Dichloro- phenyl)-piperidin-1- ylmethyl]-1- naphthalen-2- ylmethyl-2-phenyl- 1H-indole trifluoroacetate |
| 117 | H | 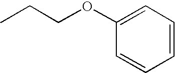 | 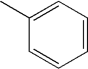 | 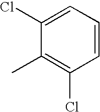 | 555 (MH⁺) | 3[4-(2,6-Dichloro- phenyl)-piperidin-1- ylmethyl]-1-(2- phenoxy-ethyl)-2- phenyl-1H-indole trifluoroacetate |
| 118 | H | 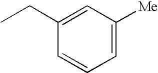 | 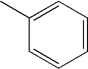 | 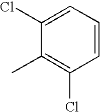 | 539 (MH⁺) | 3-[4-(2,6-Dichloro- phenyl)-piperidin-1- ylmethyl]-1-(3- methyl-benzyl)-2- phenyl-1H-indole trifluoroacetate |
| 119 | H | 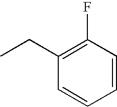 | 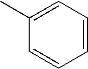 | 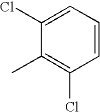 | 543 (MH⁺) | 3-[4-(2,6-Dichloro- phenyl)-piperidin-1- ylmethyl]-1-(2- fluoro-benzyl)-2- phenyl-1H-indole trifluoroacetate |
| 120 | H | 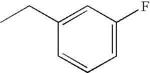 | 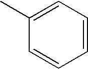 | 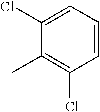 | 543 (MH⁺) | 3-[4-(2,6-Dichloro- phenyl)-piperidin-1- ylmethyl]-1-(3- fluoro-benzyl)-2- phenyl-1H-indole trifluoroacetate |
| 121 | H | 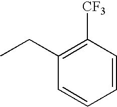 | 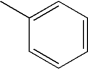 | 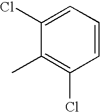 | 593 (MH⁺) | 3-[4-(2,6-Dichloro- phenyl)-piperidin-1- ylmethyl]-2-phenyl- 1-(2-trifluoromethyl- benzyl)-1H-indole trifluoroacetate |
| 122 | H | 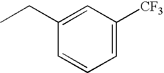 | 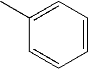 | 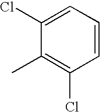 | 593 (MH⁺) | 3-[4-(2,6-Dichloro- phenyl)-piperidin-1- ylmethyl]-2-phenyl- 1-]3-trifluoromethyl- benzyl)-1H-indole trifluoroacetate |
| 123 | H | 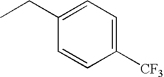 | 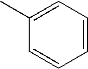 | 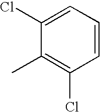 | 593 (MH⁺) | 3-[4-(2,6-Dichloro- phenyl)-piperidin-1- ylmethyl]-2-phenyl- 1-(4-trifluoromethyl- benzyl)-1H-indole trifluoroacetate |

### EXAMPLE 124

### 1-Benzenesulfonyl-3-[4-(2,6-dichloro-phenyl)-piperidin-1-ylmethyl]-2-phenyl-1H-indole trifluoroacetate

100 mg (0.229 mmol) of 3-[4-(2,6-dichloro-phenyl)-piperidin-1-ylmethyl]-2-phenyl-1H-indole were dissolved into 3 mL of a biphasic system consisting of a 1:1 mixture of toluene and 50% aqueous NaOH; two drops of Aliquat® 336 were added and the system was stirred vigorously for 15 min, then 0.037 mL of benzenesulfonyl chloride were added; two further additions of 0.015 mL of chloride were done during a period of 20 h of stirring at room temperature. The resulting mixture was poured onto Chem-elute cartridge to eliminate water and eluted with ethyl acetate; the filtrate was evaporated in vacuo and the residue was purified by preparative HPLC on a Symmetry C18 column, by gradient elution with a solvent system water/TFA 99.9:0.1 respectively (A) and CH₃CN/TFA 99.9:0.1 respectively (B) with the following gradient: 25% B (1 min); 25% B→95% B (8 min); 95% B→25% B (1 min), yielding 29 mg of the title compound.

MS (m/z): 575 (MH⁺).

Compound described in Example 125 was obtained following procedure described in Example 124.

### EXAMPLE 125

### 1-Benzoyl-3-[4-(2,6-dichloro-phenyl)-piperidin-1-ylmethyl]-2-phenyl-1H-indole trifluoroacetate

MS (m/z): 539 (MH⁺).

### Preparation 1

### 4-(2,6-Dichloro-phenyl)-1-(indole-2-carbonyl)-piperidine

A solution of 2 g (12.4 mmol) of 1H-indole-2-carboxylic acid in 80 mL of THF was cooled to 0° C.; 1.9 g (12.4 mmol) of 1-hydroxybenzotriazole and 2.38 g (12.4 mmol) of N′-(3-dimethylaminopropyl)-N′-ethylcarbodiimide hydrochloride were added and the reaction mixture was stirred at room temperature overnight. A solution of 2.57 g (11.2 mmol) of 4-(2,6-dichloro-phenyl)-piperidine in 20 mL of THF was added and after 48 h, water was added followed by Et₂O. The organic phase was collected, washed twice with saturated NaHCO₃ solution, dried with Na₂SO₄ and the solvent was removed in vacuo. The resulting residue was triturated with (i-Pr)₂O, yielding 3.73 g of the title compound.

MS (m/z): 373 (MH⁺).

### EXAMPLE 126

### 2-[4-(2,6-Dichloro-phenyl)-piperidin-1-ylmethyl]-1H-indole hydrochloride

A solution of 2 g (5.3 mmol) of 4-(2,6-dichloro-phenyl)-1-(indole-2-carbonyl)-piperidine in 35 mL of dry THF was cooled to 0° C. under a nitrogen atmosphere, then 30 mL of a 1M solution of borane/THF complex were added dropwise. The reaction mixture was allowed to warm to room temperature, then heated to reflux for 3 h, after which time it was cooled to 0° C. and quenched by careful addition of water. Concentrated NaOH solution was added up to pH 11, then the reaction mixture was heated to reflux for 5 h. After cooling, water was added and the reaction mixture was extracted with Et₂O. The organic phase was collected, washed with brine and the solvent was removed in vacuo, yielding 2.36 g of crude product, which was dissolved in 10 mL of Et₂O, cooled to 0° C. and brought to acidic pH with Et₂O/HCl. The resulting solid was filtered and dried, yielding 1.53 g of the title compound.

M.p.=238-240° C. NMR (free base, 300 MHz, CDCl₃, δ ppm): 8.76 (s br, 1H); 7.55 (d, 1H); 7.37 (d, 1H); 7.27 (m, 2H); 7.15 (dd, 1H); 7.07 (dd, 1H); 7.04 dd, 1H); 6.36 (m, 1H); 3.70 (s, 2H); 3.54 (tt, 1H); 3.04 (m, 2H); 2.65 (dq, 2H); 2.15 (dt, 2H); 1.57 (m, 2H). MS (m/z): 358 (M⁺.); 228; 130.

### EXAMPLE 127

### 2-[4-(2,6-Dichloro-phenyl)-piperidin-1-ylmethyl]-3-methyl-1H-indole

A solution of 475 mg (2.07 mmol) of 4-(2,6-dichloro-phenyl)-piperidine in 4 mL of MeOH was added to a solution of 100 mg (0.63 mmol) of 3-methyl-1H-indole-2-carbaldehyde in 2 mL of MeOH. After stirring for 1 h at room temperature, 43.6 mg (0.692 mmol) of NaBH₃CN were added, followed by one drop of AcOH. After stirring for 20 h, 22 mg of NaBH₃CN were added and the reaction mixture was stirred for additional 16 h. The solid was filtered out and the solvent was removed in vacuo. The resulting residue was taken up in AcOEt, water was added and after exhaustive extraction the organic phase was collected, dried over Na₂SO₄ and the solvent removed in vacuo. The resulting crude product was purified by chromatography, eluting with a mixture hexane/AcOEt 8:2 respectively, obtaining 25 mg of the title product.

M.p.=139-141° C. NMR (300 MHz, CDCl₃, δ ppm): 8.37 (s br, 1H); 7.51 (d, 1H); 7.33 (d, 1H); 7.27 (m, 2H); 7.15 (dd, 1H); 7.08 (dd, 1H); 7.04 (dd, 1H); 3.66 (s, 2H); 3.54 (tt, 1H); 3.02 (m, 2H); 2.64 (dq, 2H); 2.28 (s, 3H); 2.15 (dt, 2H); 1.56 (m, 2H). MS (m/z): 372 (M⁺.); 230; 143.

### EXAMPLE 128

### 2-[4-(2,6-Dimethyl-phenyl)-piperidin-1-ylmethyl]-1H-indole

A mixture of 70 mg (0.482 mmol) of 1H-indole-2-carbaldehyde, 91 mg (0.402 mmol) of 4-(2,6-dimethyl-phenyl)-piperidine hydrochloride, 415 mg of polymer supported cyanoborohydride and 10 drops of AcOH in 4 mL of THF was stirred 15 h at room temperature. The polymer was filtered out and washed with MeOH (3 mL) and THF (3 mL). The resulting solution was poured onto a SCX cartridge and washed with MeOH to eliminate non-basic impurities, then with 3% methanolic ammonia solution to recover the desired product. The solvent was removed in vacuo and the resulting crude product was purified by chromatography, eluting with a mixture DCM/AcOEt 95:5 respectively, obtaining 118 mg of the title compound.

M.p.=128-130° C. NMR (300 MHz, CDCl₃, δ ppm): 8.56 (s br, 1H); 7.55 (d, 1H); 7.37 (d, 1H); 7.15 (dd, 1H); 7.08 (dd, 1H); 6.98 (s, 3H); 6.37 (s, 1H); 3.70 (s, 2H); 3.08-2.94 (m, 3H); 2.43 (s br, 6H); 2.28 (dq, 2H); 2.13 (dt, 2H); 1.63 (m, 2H). MS (m/z): 318 (M⁺.); 188; 130.

### EXAMPLE 129

### 2-[4-(2,6-Dichloro-phenyl)-piperidin-1-ylmethyl]-3-phenyl-1H-indole

A mixture of 106 mg (0.48 mmol) of 3-phenyl-1H-indole-2-carbaldehyde, 107 mg (0.4 mmol) of 4-(2,6-dichloro-phenyl)-piperidine hydrochloride, 414 mg of polymer supported cyanoborohydride and 20 drops of AcOH in 4 mL of THF and 0.5 mL of MeOH was stirred overnight. The polymer was filtered out and washed with MeOH and THF. The resulting solution was poured onto a SCX cartridge and washed with MeOH to eliminate non-basic impurities, then with 3% methanolic ammonia solution to recover the desired product. The solvent was removed in vacuo and the resulting solid was dissolved in DCM and treated with polymer supported isocyanate for 3 h, then the polymer was filtered out and the solvent was removed in vacuo, yielding 80 mg of the title product.

M.p.=162-164° C. NMR (300 MHz, CDCl₃, δ ppm): 9.76 (s br, 1H); 7.63 (d, 1H); 7.53-7.43 (m, 6H); 7.35 (m, 1H); 7.30-7.19 (m, 2H); 7.11 (dd, 1H); 7.05 (dd, 1H); 4.04 (s, 2H); 3.57 (tt, 1H); 3.18 (m, 2H); 2.78 (m, 2H); 2.35 (m, 2H); 1.60 (m, 2H). MS (m/z): 434 (M⁺.); 228; 206; 178.

### EXAMPLE 130

### 2-[4-(2-Chloro-6-fluoro-phenyl)-piperidin-1-ylmethyl]-1H-indole

50 mg (0.344 mmol) of 1H-indole-2-carbaldehyde and 71.8 mg (0.287 mmol) of 4-(2-chloro-6-fluoro-phenyl)-piperidine hydrochloride were dissolved in 4 mL of MeOH and 1 mL of a 1M solution of AcONa in MeOH. 296 mg of polymer supported cyanoborohydride were added and, after 10 min, 0.5 mL of AcOH. The reaction mixture was stirred at room temperature for 60 h, then the polymer was filtered out and washed with MeOH and THF. The resulting solution was poured onto a SCX cartridge and washed with MeOH to eliminate non-basic impurities, then with 3% methanolic ammonia solution to recover the desired product. The solvent was removed in vacuo, yielding 90 mg of the title compound.

MS (m/z): 343 (MH⁺)

Compounds of formula (3) and described in Table 3 were obtained according to procedure described in Example 130.

TABLE 3

| | |
|---|---|
| | (3) |
| 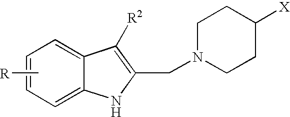 | |

| Ex. | | | | | |
|---|---|---|---|---|---|
| no | R | R² | X | MS (m/z) | Name |
| 131 | H | Me | 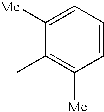 | 333 (MH⁺) | 2-[4-(2,6-Dimethyl- phenyl)-piperidin-1- ylmethyl]-3-methyl-1H- indole |
| 132 | H | Me | 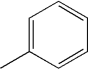 | 305 (MH⁺) | 3-Methyl-2-(4-phenyl- piperidin-1-ylmethyl)- 1H-indole |
| 133 | H | Ph | 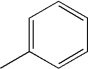 | 367 (MH⁺) | 3-Phenyl-2-(4-phenyl- piperidin-1-ylmethyl)- 1H-indole |
| 134 | H | Ph | 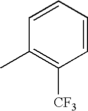 | 435 (MH⁺) | 3-Phenyl-2-(4-(3- trifluoromethylphenyl)- piperidin-1-ylmethyl)-1H- indole |
| 135 | H | Ph | 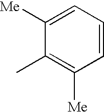 | 395 (MH⁺) | 2-[4-(2,6-Dimethyl- phenyl)-piperidin-1- ylmethyl]-3-phenyl-1H- indole |
| 136 | H | H | 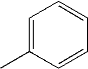 | 291 (MH⁺) | 2-(4-Phenyl-piperidin-1- ylmethyl)-1H-indole |
| 137 | H | H | 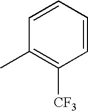 | 359 (MH⁺) | 2-[4-(2-Trifluoromethyl- phenyl)-piperidin-1- ylmethyl]-1H-indole |
| 138 | H | H | 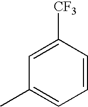 | 359 (MH⁺) | 2-[4-(3-Trifluoromethyl- phenyl)-piperidin-1- ylmethyl]-1H-indole |
| 139 | H | H | 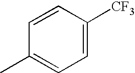 | 359 (MH⁺) | 2-[4-(4-Trifluoromethyl- phenyl)-piperidin-1- ylmethyl]-1H-indole |
| 140 | H | H | 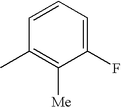 | 323 (MH⁺) | 2-[4-(3-Fluoro-2- methyl-phenyl)- piperidin-1-ylmethyl]- 1H-indole |
| 141 | 5,6-Cl₂ | H | 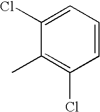 | 427 (MH⁺) | 5,6-Dichloro-2-[4-(2,6- dichloro-phenyl)- piperidin-1-ylmethyl]- 1H-indole |
| 142 | 5,6-Cl₂ | H | 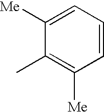 | 387 (MH⁺) | 5,6-Dichloro-2-[4-(2,6- dimethyl-phenyl)- piperidin-1-ylmethyl]- 1H-indole |

### EXAMPLE 143

### 1-Benzyl-2-[4-(2,6-dichloro-phenyl)-piperidin-1-ylmethyl]-1H-indole hydrochloride

70 mg (0.19 mmol) of 2-[4-(2,6-dichloro-phenyl)-piperidin-1-ylmethyl]-1H-indole were dissolved into 5 mL of a biphasic system consisting of a 2:1 mixture of THF and 50% aqueous NaOH; 2 mg of tetrabutylammonium bromide, followed by 25 μL (0.21 mmol) of benzylbromide were added and the system was stirred vigorously for 4 h, under nitrogen, at room temperature; one further addition of 25.5 μL of benzylbromide was done and the solution was stirred for 1 h. The resulting mixture was diluted with ethyl acetate and washed first with water and then with brine; the organic phase was dried with Na₂SO₄ and evaporated in vacuo and the residue was purified by flash-chromatography (Hexane:Et₂O=20:1 respectively) and the product was treated with ether saturated with HCl to obtain 20 mg of the title compound.

NMR (free base, 300 MHz, CDCl₃, δ ppm): 7.58 (d, 1H); 7.29-7.16 (m, 6H); 7.15-7.03 (m, 4H); 7.02 (dd, 1H); 6.45 (s, 1H); 5.58 (s, 2H); 3.57 (s, 2H); 3.49 (tt, 1H); 3.01 (m, 2H); 2.50 (dq, 2H); 2.08 (dt, 2H); 1.49 (m, 2H). MS (m/z): 448 (M⁺.); 218; 130.

Compounds of formula (4) and described in Table 4 were obtained according to procedure described in Example 143.

TABLE 4

| | |
|---|---|
| | (4) |
| 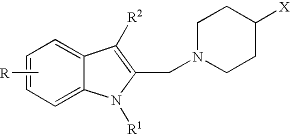 | |

| Ex. | | | | | MS | |
|---|---|---|---|---|---|---|
| no | R | R¹ | R² | X | (m/z) | Name |
| 144 | H | n-Pr | H | 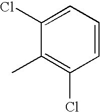 | 401 (MH⁺) | 2-[4-(2,6-Dichloro- phenyl)-piperidin-1- ylmethyl]-1-propyl- 1H-indole |
| 145 | H | Me | H | 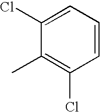 | 373 (MH⁺) | 2-[4-(2,6-Dichloro- phenyl)-piperidin-1- ylmethyl]-1-methyl- 1H-indole |

### EXAMPLE 146

### 2-(4-(2,6-Dichlorophenyl)-piperidin-1-ylmethyl)-1-(2-hydroxyethyl)-1H-indole hydrochloride

100 mg (0.28 mmol) of 2-[4-(2,6-dichloro-phenyl)-piperidin-1-ylmethyl]-1H-indole were dissolved into 4 mL of THF, then 2 mL of 50% aqueous NaOH were added and the mixture was stirred at room temperature; after 20 min 85 μL (0.56 mmol) of 2-(2-bromoethoxy)tetrahydro-2H-pyrane were added and the solution was stirred vigorously for 6 h, under nitrogen, at room temperature; one further addition of 85 μL of 2-(2-bromoethoxy)tetrahydro-2H-pyrane was done and the temperature was raised to 60° C. until complete conversion of the starting indole was observed. The resulting mixture was cooled to room temperature, diluted with ethyl ether and extracted with water; the organic phase was dried with sodium sulfate and evaporated in vacuo and the residue was purified by flash-chromatography (Hexane:Ethyl acetate=10:1 respectively); the resulting product was dissolved in 12 mL of THF, 3 mL of 1N HCl were added and the reaction mixture was stirred for 2 h at room temperature; the solution was diluted with water, then basified with 26% NH₄OH solution and extracted with Et₂O; the organic phase was dried with sodium sulfate and evaporated in vacuo to obtain 55 mg of free base, which was dissolved in Et₂O; the solution was brought to acidic pH with Et₂O/HCl and the resulting solid was filtered and dried, yielding 41 mg of the title compound.

M.p.=263-265° C. NMR (free base, 300 MHz, CDCl₃, δ ppm): 7.59 (d, 1H); 7.29-7.19 (m, 4H); 7.11 (dd, 1H); 7.02 (dd, 1H); 6.44 (s, 1H); 5.15 (s br, 1H); 4.35 (m, 2H); 4.00 (m, 2H); 3.64 (s, 2H); 3.56 (tt, 1H); 3.21 (m, 2H); 2.64 (dq, 2H); 2.21 (dt, 2H); 1.56 (m, 2H). MS (m/z): 402 (M⁺.); 372; 242; 228; 175; 145; 131.

### EXAMPLE 147

### 1-Benzoyl-2-[4-(2,6-dichloro-phenyl)-piperidin-1-ylmethyl]-1H-indole

88 mg (0.24 mmol) of 2-[4-(2,6-dichloro-phenyl)-piperidin-1-ylmethyl]-1H-indole were dissolved into 12 mL of CH₂Cl₂; 255 mg (6.37 mmol) of NaOH and 9 mg of tetrabutylammonium hydrogen sulfate were added and the suspension was stirred at room temperature; after 15 min, 31 μL (1.1 mmol) of benzoylchloride were added and the solution was stirred vigorously for 3 h, under nitrogen, at room temperature; four further additions of 31 μL of benzoylchloride were done to obtain the total conversion of the starting indole; the mixture was diluted with water and extracted with CH₂Cl₂ and the organic phase was dried with sodium sulfate, evaporated in vacuo and the residue was purified by preparative HPLC on a Symmetry C18 column, by gradient elution with a solvent system water/TFA 99.9:0.1 respectively (A) and CH₃CN/TFA 99.9:0.1 respectively (B) with the following gradient: 25% B (1 min); 25% B→95% B (8 min); 95% B→25% B (1 min), yielding 12.5 mg of the title compound.

MS (m/z): 463 (MH⁺)

Pharmacological Tests

Receptor Binding Studies

The receptor binding studies were carried out on 96-well plates; the incubation medium was Tris HCl pH 7.4 (4° C.) containing MgCl₂ (5 mM), EGTA (0.2 mM), BSA (0.1%), with a final volume of 1.0 ml, using as radioligand [³H]-AcRYYRWK-NH₂ (Amersham, 103 Ci/mmol). The samples were incubated at 37° C. for 120 min. and were then filtered off via Whatman GF/B filters pre-treated with 0.2% polyethylenimine. The filters were washed three times with Tris HCl buffer pH 7.4 (4° C.). The radioactivity present on the filters was measured using a Packard Top Count microplate scintillation counter.

The compounds of formula (I) of the present invention have a binding affinity (Ki) for the ORL-1 receptor in the range from 1 to 1000 nM.

The most potent compounds of formula (I) of the present invention have a binding affinity (Ki) to the ORL-1 receptor in the range from 1 to 100 nM.

Preparation of Membrane for the GTPγS Binding Test

The entire process was carried out at 4° C. The buffer used consisted of Tris HCl 10 mM, EDTA 0.1 mM, pH 7.4 (4° C.) (T.E.).

The cells removed from the culture flask are centrifuged at low speed to remove the growth medium.

1. Resuspend the pellets (a 175 cm² T flask in 0.5-1 ml T.E.).

2. Homogenize the cells using an Ultra-Turrax

3. Centrifuge the homogenate at 1,500 rpm for 10 min at 4° C.

4. Discard the pellets P1

5. Centrifuge the supernatant at 14,000 rpm for 30 min.

6. Discard the supernatant.

7. Resuspend the pellets P2 by suction (microsomal fraction) in 200 ml of T.E. and preserve frozen at −80° C.

To estimate the proteins, dilute the preparation 3× in T.E. and assay against standard BSA curve 0-2 mg/ml in T.E. The protein concentration is normally between 1 and 4 mg/ml. The typical yield is 1 mg of proteins per 175 cm² T flask at confluence.

Binding TESTS [³⁵S]-GTPγS

The tests were carried out in a 96-well plate using the method modified by Wieland and Jacobs (Methods Enzymol., 1994, 237, 3-13). The membranes (10 μg per well) and the SPA granules of wheat germ agglutinin (Amersham Pharmacia) (0.5 mg per well) were pre-mixed in buffer solution (HEPES 20 mM, NaCl 100 mM, MgCl₂ 10 mM, pH 7.4, 4° C.) and pre-incubated with 10 μM GDP. Increasing concentrations of the compounds to be tested were then incubated with the membrane/granule mixture for 30 min at ambient temperature. 0.3 nM [³⁵S]-GTPγS (1170 Ci/mmol, Amersham) and the ORL-1 agonist were then added. The total volume of the assay is 100 μl per well. The plates are then incubated at ambient temperature for 30 min under agitation and then centrifuged at 1500 g for 5 min. The quantity of [³⁵S]-GTPγS bound to the membranes was determined by a Wallac microbeta 1450-Trilux scintillation counter.

The activity of the compound is evaluated as inhibition of [C³⁵S]-GTPγS binding stimulation induced by the agonist.

The pIC50 values are determined as the concentration of compound which causes a 50% inhibition of the agonist response.

1. A compound of formula (I), 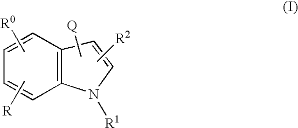
R²
or a pharmaceutically acceptable salt or solvate thereof, wherein:
R and R⁰ are each independently hydrogen, halogen, C₁₋₆alkyl, perhaloC₁₋₆alkyl, C₁₋₆alkoxy, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl, cyano and, when R and R⁰ are on adjacent carbon atoms, methylenedioxy and ethylenedioxy;
R¹ is hydrogen, C₁₋₆alkyl, C₃₋₆alkenyl, C₃₋₆alkinyl, arylC₁₋₆alkyl, heteroarylC₁₋₆alkyl, (C₃₋₇cycloalkyl)alkyl, aminoC₁₋₆alkyl, (C₁₋₆alkyl)aminoC₁₋₆alkyl, di(C₁₋₆alkyl)aminoC₁₋₆alkyl, hydroxyC₁₋₆alkyl, C₁₋₆alkoxyC₁₋₆alkyl, aryloxyC₁₋₆alkyl, CO-aryl, SO₂aryl, aryl, C₁₋₆alkolxycarbonylC₁₋₆alkyl, where each aryl or heteroaryl can be substituted one or more times by halogen, C₁₋₆alkoxy, C₁₋₆alkyl, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl or perhaloC₁₋₆alkyl;
R² is C₃₋₇cycloalkyl, aryl, heteroaryl, arylC₁₋₆alkyl, heteroarylC₁₋₆alkyl, C₁₋₆alkoxycarbonyl, hydroxyC₁₋₆alkyl, aminocarbonyl, C₁₋₆alkylaminocarbonyl, di(C₁₋₆alkyl)aminocarbonyl where each aryl or heteroaryl can be substituted one or more times by halogen, C₁₋₆alkoxy, C₁₋₆alkyl, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl or perhaloC₁₋₆alkyl;
Q is a moiety of formula: 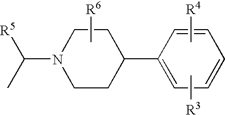
R⁶R⁴
wherein:
R³ and R⁴ are each independently hydrogen, halogen, C₁₋₆alkyl, perhaloC₁₋₆alkyl, C₁₋₆alkoxy, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl;
R⁵ is hydrogen or C₁₋₆alkyl, and
R⁶ is hydrogen or hydroxymethyl.

2. A compound of formula (I) according to claim 1, wherein R and R⁰ independently represent hydrogen, halogen, C₁₋₆alkyl, C₁₋₆alkoxy.

3. A compound of formula (I) according to claim 2, wherein R and R⁰ independently represent hydrogen, chlorine, fluorine, methyl, methoxy.

4. A compound of formula (I) according to claim 1, wherein R¹ is hydrogen, C₁₋₆alkyl, C₃₋₆alkenyl, C₃₋₆alkinyl, arylC₁₋₆alkyl, (C₃₋₇cycloalkyl)alkyl, hydroxyC₁₋₆alkyl, CO-aryl, SO₂-aryl.

5. A compound of formula (I) according to claim 4, wherein R¹ is hydrogen, methyl, n-propyl, isopentyl, allyl, 2-hydroxyethyl, cyclopropylmethyl, cyclohexylmethyl, benzyl, fluorobenzyl, chlorobenzyl, bromobenzyl, methoxybenzyl, methylbenzyl, t-butylbenzyl, trifluoromethylbenzyl, diphenylmethyl, phenoxyethyl, 2-naphthylmethyl, benzoyl, benzenesulfonyl.

6. A compound of formula (I) according to claim 1, wherein R² is aryl, heteroaryl, arylC₁₋₆alkyl, C₁₋₆alkoxycarbonyl.

7. A compound of formula (I) according to claim 6, wherein R² is phenyl, chlorophenyl, methoxyphenyl, fluorophenyl, 2-furyl, 2-thienyl, 2-pyridyl, benzyl, ethoxycarbonyl.

8. A compound of formula (I) according to claim 1, wherein R³ and R⁴ independently represent hydrogen, halogen, C₁₋₆alkyl, perhaloC₁₋₆alkyl, C₁₋₆alkoxy.

9. A compound of formula (I) according to claim 8, wherein R³ and R⁴ independently represent hydrogen, chlorine, fluorine, bromine, methyl, methoxy, trifluoromethyl.

10. A compound of formula (I) according to claim 1, wherein R⁵ and R⁶ represent hydrogen.

11. A compound of formula (I) according to claim 1, or a pharmaceutically acceptable salt or solvate thereof, selected from:
3-[4-(2,6-Dichloro-phenyl)-piperidin-1-ylmethyl]-2-phenyl-1H-indole;
3-[4-(2,6-Dichloro-phenyl)-piperidin-1-ylmethyl]-1H-indole;
3-[4-(2,6-Dichloro-phenyl)-piperidin-1-ylmethyl]-2-methyl-1H-indole;
2-(4-Chloro-phenyl)-3-[4-(2,6-dimethyl-phenyl)-piperidin-1-ylmethyl]-1H-indole;
2-Phenyl-3-[4-(2-trifluoromethyl-phenyl)-piperidin-1-ylmethyl]-1H-indole;
3-[4-(2,6-Dimethyl-phenyl)-piperidin-1-ylmethyl]-2-phenyl-1H-indole;
2-Phenyl-3-(4-phenyl-piperidin-1-ylmethyl)-1H-indole;
2-(2-Chloro-phenyl)-3-[4-(2,6-dichloro-phenyl)-piperidin-1-ylmethyl]-1H-indole;
2-(2-Chloro-phenyl)-3-[4-(2,6-dimethyl-phenyl)-piperidin-1-ylmethyl]-1H-indole;
2-(2-Chloro-phenyl)-3-[4-(2-trifluoromethyl-phenyl)-piperidin-1-ylmethyl]-1H-indole;
2-(2-Chloro-phenyl)-3-(4-phenyl-piperidin-1-ylmethyl)-1H-indole;
3-[4-(2,6-Dichloro-phenyl)-piperidin-1-ylmethyl]-2-(2-methoxy-phenyl)-1H-indole;
3-[4-(2,6-Dimethyl-phenyl)-piperidin-1-ylmethyl]-2-(2-methoxy-phenyl)-1H-indole;
2-(2-Methoxy-phenyl)-3-[4-(2-trifluoromethyl-phenyl)-piperidin-1-ylmethyl]-1H-indole;
2-(2-Methoxy-phenyl)-3-(4-phenyl-piperidin-1-ylmethyl)-1H-indole;
3-[4-(2,6-Dichloro-phenyl)-piperidin-1-ylmethyl]-2-(3-methoxy-phenyl)-1H-indole;
3-[4-(2,6-Dimethyl-phenyl)-piperidin-1-ylmethyl]-2-(3-methoxy-phenyl)-1H-indole;
2-(3-Methoxy-phenyl)-3-[4-(2-trifluoromethyl-phenyl)-piperidin-1-ylmethyl]-1H-indole;
2-(3-Methoxy-phenyl)-3-(4-phenyl-piperidin-1-ylmethyl)-1H-indole;
2-(4-Chloro-phenyl)-3-[4-(2,6-dichloro-phenyl)-piperidin-1-ylmethyl]-1H-indole;
2-(4-Chloro-phenyl)-3-[4-(2-trifluoromethyl-phenyl)-piperidin-1-ylmethyl]-1H-indole;
2-(4-Chloro-phenyl)-3-(4-phenyl-piperidin-1-ylmethyl)-1H-indole;
3-[4-(2,6-Dichloro-phenyl)-piperidin-1-ylmethyl]-2-(4-fluoro-phenyl)-1H-indole;
3-[4-(2,6-Dimethyl-phenyl)-piperidin-1-ylmethyl]-2-(4-fluoro-phenyl)-1H-indole;
2-(4-Fluoro-phenyl)-3-[4-(2-trifluoromethyl-phenyl)-piperidin-1-ylmethyl]-1H-indole;
2-(4-Fluoro-phenyl)-3-(4-phenyl-piperidin-1-ylmethyl)-1H-indole;
3-[4-(2,6-Dichloro-phenyl)-piperidin-1-ylmethyl]-2-furan-2-yl-1H-indole;
3-[4-(2,6-Dimethyl-phenyl)-piperidin-1-ylmethyl]-2-furan-2-yl-1H-indole;
2-Furan-2-yl-3-[4-(2-trifluoromethyl-phenyl)-piperidin-1-ylmethyl]-1H-indole;
2-Furan-2-yl-3-(4-phenyl-piperidin-1-ylmethyl)-1H-indole;
3-[4-(2,6-Dichloro-phenyl)-piperidin-1-ylmethyl]-2-pyridin-2-yl-1H-indole;
3-[4-(2,6-Dimethyl-phenyl)-piperidin-1-ylmethyl]-2-pyridin-2-yl-1H-indole;
2-Pyridin-2-yl-3-[4-(2-trifluoromethyl-phenyl)-piperidin-1-ylmethyl]-1H-indole;
3-(4-Phenyl-piperidin-1-ylmethyl)-2-pyridin-2-yl-1H-indole;
3-[4-(2,6-Dichloro-phenyl)-piperidin-1-ylmethyl]-2-thiophen-2-yl-1H-indole;
3-[4-(2,6-Dimethyl-phenyl)-piperidin-1-ylmethyl]-2-thiophen-2-yl-1H-indole;
2-Thiophen-2-yl-3-[4-(2-trifluoromethyl-phenyl)-piperidin-1-ylmethyl]-1H-indole;
3-(4-Phenyl-piperidin-1-ylmethyl)-2-thiophen-2-yl-1H-indole;
2-Benzyl-3-[4-(2,6-dimethyl-phenyl)-piperidin-1-ylmethyl]-1H-indole;
2-Benzyl-3-[4-(2,6-dichloro-phenyl)-piperidin-1-ylmethyl]-1H-indole;
3-[4-(4-Methoxy-phenyl)-piperidin-1-ylmethyl]-2-phenyl-1H-indole;
3-[4-(2-Fluoro-phenyl)-piperidin-1-ylmethyl]-2-phenyl-1H-indole;
3-[4-(3-Fluoro-phenyl)-piperidin-1-ylmethyl]-2-phenyl-1H-indole;
3-[4-(4-Fluoro-phenyl)-piperidin-1-ylmethyl]-2-phenyl-1H-indole;
2-Phenyl-3-[4-(4-trifluoromethyl-phenyl)-piperidin-1-ylmethyl]-1H-indole;
3-[4-(2-Chloro-phenyl)-piperidin-1-ylmethyl]-2-phenyl-1H-indole;
3-[4-(3-Chloro-phenyl)-piperidin-1-ylmethyl]-2-phenyl-1H-indole;
3-[4-(4-Chloro-phenyl)-piperidin-1-ylmethyl]-2-phenyl-1H-indole;
2-Phenyl-3-(4-o-tolyl-piperidin-1-ylmethyl)-1H-indole;
3-[4-(2-Bromo-phenyl)-piperidin-1-ylmethyl]-2-phenyl-1H-indole;
3-[4-(2,3-Dichloro-phenyl)-piperidin-1-ylmethyl]-2-phenyl-1H-indole;
3-[4-(2,5-Dimethyl-phenyl)-piperidin-1-ylmethyl]-2-phenyl-1H-indole;
3-[4-(2,6-Difluoro-phenyl)-piperidin-1-ylmethyl]-2-phenyl-1H-indole;
3-[4-(3-Bromo-phenyl)-piperidin-1-ylmethyl]-2-phenyl-1H-indole;
3-[4-(2-Methoxy-phenyl)-piperidin-1-ylmethyl]-2-phenyl-1H-indole;
3-[4-(2,6-Dichloro-phenyl)-piperidin-1-ylmethyl]-5-fluoro-2-phenyl-1H-indole;
3-[4-(2,6-Dimethyl-phenyl)-piperidin-1-ylmethyl]-1H-indole;
3-[4-(2,6-Dimethyl-phenyl)-piperidin-1-ylmethyl]-2-methyl-1H-indole;
Cis-[4-Phenyl-1-(2-phenyl-1H-indol-3-ylmethyl)-piperidin-3-yl]-methanol;
Trans-[4-Phenyl-1-(2-phenyl-1H-indol-3-ylmethyl)-piperidin-3-yl]-methanol;
5-Chloro-3-[4-(2,6-dimethyl-phenyl)-piperidin-1-ylmethyl]-2-phenyl-1H-indole;
3-[4-(2,6-Dimethyl-phenyl)-piperidin-1-ylmethyl]-5-methoxy-2-phenyl-1H-indole;
7-[4-(2,6-Dimethyl-phenyl)-piperidin-1-ylmethyl]-6-phenyl-5H-[1,3]dioxolo[4,5-f]indole;
3-[4-(2,6-Dimethyl-phenyl)-piperidin-1-ylmethyl]-1-(2-hydroxy-ethyl)-2-phenyl-1H-indol-5-ol;
7-Bromo-3-[4-(2,6-dimethyl-phenyl)-piperidin-1-ylmethyl]-2-methyl-1H-indole;
3-[4-(2,6-Dimethyl-phenyl)-piperidin-1-ylmethyl]-5-fluoro-2-methyl-1H-indole;
3-[4-(2,6-Dimethyl-phenyl)-piperidin-1-ylmethyl]-5-fluoro-2-phenyl-1H-indole;
3-[4-(2,6-Dichloro-phenyl)-piperidin-1-ylmethyl]-1H-indole-2-carboxylic acid ethyl ester;
3-[4-(2,6-Dimethyl-phenyl)-piperidin-1-ylmethyl]-2-phenyl-1H-indole-6-carbonitrile;
3-[4-(2,6-Dimethyl-phenyl)-piperidin-1-ylmethyl]-1,2-diphenyl-1H-indole;
3-[4-(2,6-Dimethyl-phenyl)-piperidin-1-ylmethyl]-5-fluoro-1H-indole-2-carboxylic acid amide trifluoroacetate;
3-{1-[4-(2,6-Dimethyl-phenyl)-piperidin-1-yl]-ethyl}-1H-indole;
{3-[4-(2,6-Dichloro-phenyl)-piperidin-1-ylmethyl]-1H-indol-2-yl}-methanol;
1-Benzyl-3-[4-(2,6-dichloro-phenyl)-piperidin-1-ylmethyl]-2-phenyl-1H-indole;
3-[4-(2,6-Dichloro-phenyl)-piperidin-1-ylmethyl]-2-phenyl-1-propyl-1H-indole;
3-[4-(2,6-Dichloro-phenyl)-piperidin-1-ylmethyl]-1-methyl-2-phenyl-1H-indole;
1-Benzyl-3-[4-(2,6-dichloro-phenyl)-piperidin-1-ylmethyl]-5-fluoro-2-phenyl-1H-indole;
1-Benzyl-3-[4-(2,6-dichloro-phenyl)-piperidin-1-ylmethyl]-2-methyl-1H-indole;
1-Benzyl-3-[4-(2,6-dichloro-phenyl)-piperidin-1-ylmethyl]-1H-indole;
1-Benzyl-3-[4-(2,6-dimethyl-phenyl)-piperidin-1-ylmethyl]-2-methyl-1H-indole;
1-Benzyl-3-[4-(2,6-dimethyl-phenyl)-piperidin-1-ylmethyl]-2-phenyl-1H-indole;
1-Benzyl-3-[4-(2,6-dimethyl-phenyl)-piperidin-1-ylmethyl]-1H-indole;
1-Benzyl-5-chloro-3-[4-(2,6-dimethyl-phenyl)-piperidin-1-ylmethyl]-2-phenyl-1H-indole;
1-Benzyl-3-[4-(2,6-dimethyl-phenyl)-piperidin-1-ylmethyl]-5-methoxy-2-phenyl-1H-indole;
5-Benzyl-7-[4-(2,6-dimethyl-phenyl)-piperidin-1-ylmethyl]-6-phenyl-5H-[1,3]dioxolo[4,5-f]indole;
{3-[4-(2,6-Dichloro-phenyl)-piperidin-1-ylmethyl]-5-fluoro-2-phenyl-indol-1-yl}-acetic acid methyl ester;
3-(4-(2,6-Dichloro-phenyl)piperidin-1-ylmethyl)-1-(2-hydroxyethyl)-2-phenyl-1H-indole;
2-{3-[4-(2,6-Dichloro-phenyl)-piperidin-1-ylmethyl]-5-fluoro-2-phenyl-indol-1-yl}-ethanol;
2-{3-[4-(2,6-Dichloro-phenyl)-piperidin-1-ylmethyl]-indol-1-yl}-ethanol;
2-{3-[4-(2,6-Dimethyl-phenyl)-piperidin-1-ylmethyl]-indol-1-yl}-ethanol;
2-{3-[4-(2,6-Dimethyl-phenyl)-piperidin-1-ylmethyl]-2-methyl-indol-1-yl}-ethanol;
2-{3-[4-(2,6-Dichloro-phenyl)-piperidin-1-ylmethyl]-2-methyl-indol-1-yl}-ethanol;
2-{3-[4-(2,6-Dimethyl-phenyl)-piperidin-1-ylmethyl]-2-phenyl-indol-1-yl}-ethanol;
3-{3-[4-(2,6-Dimethyl-phenyl)-piperidin-1-ylmethyl]-2-phenyl-indol-1-yl}-propan-1-ol;
2-{3-[4-(2,6-Dimethyl-phenyl)-piperidin-1-ylmethyl]-5-methoxy-2-phenyl-indol-1-yl}-ethanol;
2-{5-Chloro-3-[4-(2,6-dimethyl-phenyl)-piperidin-1-ylmethyl]-2-phenyl-indol-1-yl}-ethanol;
2-{7-[4-(2,6-Dimethyl-phenyl)-piperidin-1-ylmethyl]-6-phenyl-[1,3]dioxolo[4,5-f]indol-5-yl}-ethanol;
2-{3-[4-(2,6-Dimethyl-phenyl)-piperidin-1-ylmethyl]-5-fluoro-2-methyl-indol-1-yl}-ethanol;
1-(4-tert-Butyl-benzyl)-3-[4-(2,6-dichloro-phenyl)-piperidin-1-ylmethyl]-2-phenyl-1H-indole trifluoroacetate;
3-[4-(2,6-Dichloro-phenyl)-piperidin-1-ylmethyl]-1-(3-methyl-butyl)-2-phenyl-1H-indole;
1-Cyclopropylmethyl-3-[4-(2,6-dichloro-phenyl)-piperidin-1-ylmethyl]-2-phenyl-1H-indole;
3-[4-(2,6-Dichloro-phenyl)-piperidin-1-ylmethyl]-1-(3-methoxy-benzyl)-2-phenyl-1H-indole;
3-[4-(2,6-Dichloro-phenyl)-piperidin-1-ylmethyl]-1-(2-methyl-benzyl)-2-phenyl-1H-indole;
1-Cyclohexylmethyl-3-[4-(2,6-dichloro-phenyl)-piperidin-1-ylmethyl]-2-phenyl-1H-indole;
3-[4-(2,6-Dichloro-phenyl)-piperidin-1-ylmethyl]-1-(4-methyl-benzyl)-2-phenyl-1H-indole;
3-[4-(2,6-Dichloro-phenyl)-piperidin-1-ylmethyl]-1-(4-fluoro-benzyl)-2-phenyl-1H-indole;
1-(3-Chloro-benzyl)-3-[4-(2,6-dichloro-phenyl)-piperidin-1-ylmethyl]-2-phenyl-1H-indole trifluoroacetate;
1-(2-Chloro-benzyl)-3-[4-(2,6-dichloro-phenyl)-piperidin-1-ylmethyl]-2-phenyl-1H-indole;
1-(4-Chloro-benzyl)-3-[4-(2,6-dichloro-phenyl)-piperidin-1-ylmethyl]-2-phenyl-1H-indole;
1-Allyl-3-[4-(2,6-dichloro-phenyl)-piperidin-1-ylmethyl]-2-phenyl-1H-indole;
3-[4-(2,6-Dichloro-phenyl)-piperidin-1-ylmethyl]-2-phenyl-1-prop-2-ynyl-1H-indole;
3-[4-(2,6-Dichloro-phenyl)-piperidin-1-ylmethyl]-1-(2-methoxy-benzyl)-2-phenyl-1H-indole trifluoroacetate;
3-[4-(2,6-Dichloro-phenyl)-piperidin-1-ylmethyl]-1-(4-methoxy-benzyl)-2-phenyl-1H-indole trifluoroacetate;
1-(4-Bromo-benzyl)-3-[4-(2,6-dichloro-phenyl)-piperidin-1-ylmethyl]-2-phenyl-1H-indole trifluoroacetate;
1-Biphenyl-4-ylmethyl-3-[4-(2,6-dichloro-phenyl)-piperidin-1-ylmethyl]-2-phenyl-1H-indole trifluoroacetate;
3-[4-(2,6-Dichloro-phenyl)-piperidin-1-ylmethyl]-1-naphthalen-2-ylmethyl-2-phenyl-1H-indole trifluoroacetate;
3-[4-(2,6-Dichloro-phenyl)-piperidin-1-ylmethyl]-1-(2-phenoxy-ethyl)-2-phenyl-1H-indole trifluoroacetate;
3-[4-(2,6-Dichloro-phenyl)-piperidin-1-ylmethyl]-1-(3-methyl-benzyl)-2-phenyl-1H-indole trifluoroacetate;
3-[4-(2,6-Dichloro-phenyl)-piperidin-1-ylmethyl]-1-(2-fluoro-benzyl)-2-phenyl-1H-indole trifluoroacetate;
3-[4-(2,6-Dichloro-phenyl)-piperidin-1-ylmethyl]-1-(3-fluoro-benzyl)-2-phenyl-1H-indole trifluoroacetate;
3-[4-(2,6-Dichloro-phenyl)-piperidin-1-ylmethyl]-2-phenyl-1-(2-trifluoromethyl-benzyl)-1H-indole trifluoroacetate;
3-[4-(2,6-Dichloro-phenyl)-piperidin-1-ylmethyl]-2-phenyl-1-(3-trifluoromethyl-benzyl)-1H-indole trifluoroacetate;
3-[4-(2,6-Dichloro-phenyl)-piperidin-1-ylmethyl]-2-phenyl-1-(4-trifluoromethyl-benzyl)-1H-indole trifluoroacetate;
1-Benzenesulfonyl-3-[4-(2,6-dichloro-phenyl)-piperidin-1-ylmethyl]-2-phenyl-1H-indole trifluoroacetate;
1-Benzoyl-3-[4-(2,6-dichloro-phenyl)-piperidin-1-ylmethyl]-2-phenyl-1H-indole trifluoroacetate;
2-[4-(2,6-Dichloro-phenyl)-piperidin-1-ylmethyl]-1H-indole;
2-[4-(2,6-Dichloro-phenyl)-piperidin-1-ylmethyl]-3-methyl-1H-indole;
2-[4-(2,6-Dimethyl-phenyl)-piperidin-1-ylmethyl]-1H-indole;
2-[4-(2,6-Dichloro-phenyl)-piperidin-1-ylmethyl]-3-phenyl-1H-indole;
2-[4-(2-Chloro-6-fluoro-phenyl)-piperidin-1-ylmethyl]-1H-indole;
2-[4-(2,6-Dimethyl-phenyl)-piperidin-1-ylmethyl]-3-methyl-1H-indole;
3-Methyl-2-(4-phenyl-piperidin-1-ylmethyl)-1H-indole;
3-Phenyl-2-(4-phenyl-piperidin-1-ylmethyl)-1H-indole;
3-Phenyl-2-(4-(3-trifluoromethylphenyl)piperidin-1-ylmethyl)-1H-indole;
2-[4-(2,6-Dimethyl-phenyl)-piperidin-1-ylmethyl]-3-phenyl-1H-indole;
2-(4-Phenyl-piperidin-1-ylmethyl)-1H-indole;
2-[4-(2-Trifluoromethyl-phenyl)-piperidin-1-ylmethyl]-1H-indole;
2-[4-(3-Trifluoromethyl-phenyl)-piperidin-1-ylmethyl]-1H-indole;
2-[4-(4-Trifluoromethyl-phenyl)-piperidin-1-ylmethyl]-1H-indole;
2-[4-(3-Fluoro-2-methyl-phenyl)-piperidin-1-ylmethyl]-1H-indole;
5,6-Dichloro-2-[4-(2,6-dichloro-phenyl)-piperidin-1-ylmethyl]-1H-indole;
5,6-Dichloro-2-[4-(2,6-dimethyl-phenyl)-piperidin-1-ylmethyl]-1H-indole;
1-Benzyl-2-[4-(2,6-dichloro-phenyl)-piperidin-1-ylmethyl]-1H-indole;
2-[4-(2,6-Dichloro-phenyl)-piperidin-1-ylmethyl]-1-propyl-1H-indole;
2-[4-(2,6-Dichloro-phenyl)-piperidin-1-ylmethyl]-1-methyl-1H-indole;
2-(4-(2,6-Dichlorophenyl)-piperidin-1-ylmethyl)-1-(2-hydroxyethyl)-1H-indole;
1-Benzoyl-2-[4-(2,6-dichloro-phenyl)-piperidin-1-ylmethyl]-1H-indole.

12. An enantiomer of a compound of formula (I) as described in claim 1.

13. A mixture of enantiomers of a compound of formula (I) as described in claim 1, where an enantiomer is present in greater proportion than its antipod.

14. A compound of formula (I) as defined in claim 1, for use as active therapeutic substance.

15. A pharmaceutical composition comprising a compound of formula (I) as defined in claim 1, or a pharmaceutically acceptable salt or solvate thereof, and a pharmaceutically acceptable carrier therefore.

16. A process for preparing a compound of formula (I) as defined in claim 1, comprising the step of reacting a compound of formula (III) 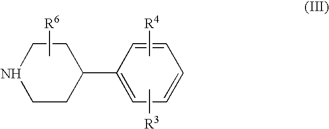
R⁶R⁴
R³
I)
wherein R³, R⁴, R⁶ are as defined as in formula (I) of claim 1, with a compound of formula (VII), 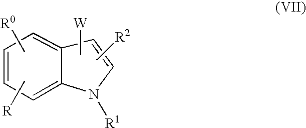
R²
R¹
R
(VII)
wherein R, R⁰, R¹, R², are as defined as in formula (I) of claim 1 and W is hydrogen or a group capable of binding to the piperidinic nitrogen of said compound of formula (III).

17. A process according to claim 16 wherein the reaction between (VII) and (III) is a Mannich reaction, taking place in an organic solvent environment, in presence of a suitable aldehydic reagent and acetic acid.

18. A process according to claim 16, wherein W is formyl, acyl or carboxyl, and the compound resulting from the reaction of (VII) with (III) is further treated with a reducing agent, thus obtaining said compound of formula (I), or the reaction of (VII) with (III) is a performed under reductive amination conditions, leading directly to said compound of formula (I).

19. A process according to claim 16, wherein R¹ in formula (VII) is hydrogen, further comprising the step of treating said compound (VII) or a derivative thereof, with a reagent of formula R¹—X where R¹ is defined as in claim 1 and X is a suitable leaving group.

20. A process according to claim 19, where said reaction with R¹—X takes place in basic conditions, or under phase transfer conditions.

21. Use of a compound of formula (VI) 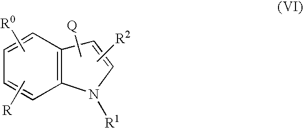
R¹
(VI)
wherein:
R and R⁰ are each independently hydrogen, halogen, C₁₋₆alkyl, perhaloC₁₋₆alkyl, C₁₋₆alkoxy, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl, cyano and, when R and R⁰ are on adjacent carbon atoms, methylenedioxy and ethylenedioxy;
R¹ is hydrogen, C₁₋₆alkyl, C₃₋₆alkenyl, C₃₋₆alkinyl, arylC₁₋₆alkyl, heteroarylC₁₋₆alkyl, (C₃₋₇cycloalkyl)alkyl, aminoC₁₋₆alkyl, (C₁₋₆alkyl)aminoC₁₋₆alkyl, di(C₁₋₆alkyl)aminoC₁₋₆alkyl, hydroxyC₁₋₆alkyl, C₁₋₆alkoxyC₁₋₆alkyl, aryloxyC₁₋₆alkyl, CO-aryl, SO₂aryl, aryl, C₁₋₆alkolxycarbonylC₁₋₆alkyl, where each aryl or heteroaryl can be substituted one or more times by halogen, C₁₋₆alkoxy, C₁₋₆alkyl, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl or perhaloC₁₋₆alkyl;
R² is hydrogen, C₁₋₆alkyl, C₃₋₇cycloalkyl, aryl, heteroaryl, arylC₁₋₆alkyl, heteroarylC₁₋₆alkyl, C₁₋₆alkoxycarbonyl, hydroxyC₁₋₆alkyl, aminocarbonyl, C₁₋₆alkylaminocarbonyl, di(C₁₋₆alkyl)aminocarbonyl where each aryl or heteroaryl can be substituted one or more times by halogen, C₁₋₆alkoxy, C₁₋₆alkyl, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl or perhaloC₁₋₆alkyl;
Q is a moiety of formula: 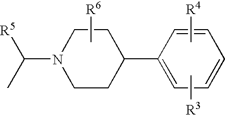
R⁶R⁴
wherein:
R³ and R⁴ are each independently hydrogen, halogen, C₁₋₆alkyl, perhaloC₁₋₆alkyl, C₁₋₆alkoxy, hydroxy, amino, C₁₋₆alkylamino, di(C₁₋₆alkyl)amino, aminoC₁₋₆alkyl, (C₁₋₆alkyl)aminoC₁₋₆alkyl, di(C₁₋₆alkyl)aminoC₁₋₆alkyl, aryl;
R⁵ is hydrogen or C₁₋₆alkyl, and
R⁶ is hydrogen or hydroxymethyl,
in the manufacture of a medicament for administration to a human or animal patient for modulating the activity of the ORL-1 receptors.

22. Use according to claim 21, wherein said drug is useful in the prophylaxis and treatment of illnesses dependent on modulation of the ORL-1 receptor.
